(19) United States
(12) Patent Application Publication
(10) Pub. No.: US 2009/0068653 A1
(43) Pub. Date: Mar. 12, 2009

(54) BIOCHEMICAL AND GENETIC ANALYSIS FOR PREDICTION OF BREAST CANCER RISK

(76) Inventors: Fritz F. Parl, Nashville, TN (US); Philip S. Crooke, Nashville, TN (US); Marylyn D. Ritchie, Brentwood, TN (US); David L. Hachey, Brentwood, TN (US); Sheila Dawling, Nolensville, TN (US); Nady Roodi, Nashville, TN (US)

Correspondence Address: FULBRIGHT & JAWORSKI L.L.P., 600 CONGRESS AVE., SUITE 2400, AUSTIN, TX, 78701, US

(21) Appl. No.: 11/854,321

(22) Filed: Sep. 12, 2007

Related U.S. Application Data
(60) Provisional application No. 60/844,553, filed on Sep. 14, 2006.

(51) Int. Cl. C12Q 1/68

(52) U.S. Cl. 435/6

(57) ABSTRACT

The present invention provides new methods for the assessment of cancer risk in the general population. These methods utilize particular alleles of three selected genes, here associated with specific biochemical activities, to identify individuals with increased or decreased risk of breast cancer. Using such methods, it is possible to reallocate healthcare costs in cancer screening to patient subpopulations at increased cancer risk and to identify candidates for cancer prophylactic treatment.

The present invention claims benefit of priority to U.S. Provisional Application Ser. No. 60/844,553, filed Sep. 14, 2006, the entire contents of which are hereby incorporated by reference.

### GOVERNMENTAL SUPPORT CLAUSE

This invention was made with government support under 1R01CA ES83752, 5P30 CA68485 and 5P30 ES00267 awarded by National Institutes of Health. The government has certain rights in the invention.

### BACKGROUND OF THE INVENTION

1. Field of the Invention

The present invention relates generally to the fields of oncology and genetics. More particularly, it concerns use of biochemical and genetic profiles of specifics alleles of three different genes to predict risk of breast cancer. These risk factors alleles, when used to screen patient samples, provide a means to direct patients towards their most effective prediagnostic cancer risk management.

2. Description of Related Art

For patients with cancer, early diagnosis and treatment are the keys to better outcomes. In 2001, there are expected to be 1.25 million persons diagnosed with cancer in the United States. Tragically, in 2001 over 550,000 people are expected to die of cancer. To a very large extent, the difference between life and death for a cancer patient is determined by the stage of the cancer when the disease is first detected and treated. For those patients whose tumors are detected when they are relatively small and confined, the outcomes are usually very good. Conversely, if a patient's cancer has spread from its organ of origin to distant sites throughout the body, the patient's prognosis is very poor regardless of treatment. The problem is that tumors that are small and confined usually do not cause symptoms. Therefore, to detect these early stage cancers, it is necessary to screen or examine people without symptoms of illness. In such apparently healthy people, cancers are actually quite rare. Therefore it is necessary to screen a large number of people to detect a small number of cancers. As a result, cancer-screening tests are relatively expensive to administer in terms of the number of cancers detected per unit of healthcare expenditure.

A related problem in cancer screening is derived from the reality that no screening test is completely accurate. All tests deliver, at some rate, results that are either falsely positive (indicate that there is cancer when there is no cancer present) or falsely negative (indicate that no cancer is present when there really is a tumor present). Falsely positive cancer screening test results create needless healthcare costs because such results demand that patients receive follow-up examinations, frequently including biopsies, to confirm that a cancer is actually present. For each falsely positive result, the costs of such follow-up examinations are typically many times the costs of the original cancer-screening test. In addition, there are intangible or indirect costs associated with falsely positive screening test results derived from patient discomfort, anxiety and lost productivity. Falsely negative results also have associated costs. Obviously, a falsely negative result puts a patient at higher risk of dying of cancer by delaying treatment. To counter this effect, it might be reasonable to increase the rate at which patients are repeatedly screened for cancer. This, however, would add direct costs of screening and indirect costs from additional falsely positive results. In reality, the decision on whether or not to offer a cancer screening test hinges on a cost-benefit analysis in which the benefits of early detection and treatment are weighed against the costs of administering the screening tests to a largely disease free population and the associated costs of falsely positive results.

Another related problem concerns the use of chemopreventative drugs for cancer. Basically, chemopreventatives are drugs that are administered to prevent a patient from developing cancer. While some chemopreventative drugs may be effective, such drugs are not appropriate for all persons because the drugs have associated costs and possible adverse side effects (Reddy and Chow, 2000). Some of these adverse side effects may be life threatening. Therefore, decisions on whether to administer chemopreventative drugs are also based on a cost-benefit analysis. The central question is whether the benefits of reduced cancer risk outweigh the costs and associated risks of the chemopreventative treatment.

Currently, an individual's age is the most important factor in determining if a particular cancer-screening test should be offered to a patient. Truly, cancer is a rare disease in the young and a fairly common ailment in the elderly. The problem arises in screening and preventing cancers in the middle years of life when cancer can have its greatest negative impact on life expectancy and productivity. In the middle years of life, cancer is still fairly uncommon. Therefore, the costs of cancer screening and prevention can still be very high relative to the number of cancers that are detected or prevented. Decisions on when to begin screening also may be influenced by personal history or family history measures. Unfortunately, appropriate informatic tools to support such decision-making are not yet available for most cancers.

A common strategy to increase the effectiveness and economic efficiency of cancer screening and chemoprevention in the middle years of life is to stratify individuals' cancer risk and focus the delivery of screening and prevention resources on the high-risk segments of the population. Two such tools to stratify risk for breast cancer are termed the Gail Model and the Claus Model (Costantino et al., 1999; McTiernan et al., 2001). The Gail model is used as the “Breast Cancer Risk-Assessment Tool” software provided by the National Cancer Institute of the National Institutes of Health on their web site. Neither of these breast cancer models utilize genetic markers as part of their inputs. Furthermore, while both models are steps in the right direction, neither the Claus nor Gail models have the desired predictive power or discriminatory accuracy to truly optimize the delivery of breast cancer screening or chemopreventative therapies.

These issues and problems could be reduced in scope or even eliminated if it were possible to stratify or differentiate a given individual's risk from cancer more accurately than is now possible. If a precise measure of actual risk could be accurately determined, it would be possible to concentrate cancer screening and chemopreventative efforts in that segment of the population that is at highest risk. With accurate stratification of risk and concentration of effort in the high-risk population, fewer screening tests would be required to detect a greater number of cancers at an earlier and more treatable stage. Fewer screening tests would mean lower test administrative costs and fewer falsely positive results. A greater number of cancers detected would mean a greater net benefit to patients and other concerned parties such as health care providers. Similarly, chemopreventative drugs would have a greater positive impact by focusing the administration of these drugs to a population that receives the greatest net benefit.

### SUMMARY OF THE INVENTION

Thus, in accordance with the present invention, there is provided a method for assessing a female subject's risk for developing breast cancer comprising (a) determining, in a sample from the subject, the allelic profile of COMT, CYP1A1 and CYP1B1; and (b) predicting, based an in silico model of estrogen biosynthesis, relative amounts of 4-OHE₂ and/or E₂-3,4-Q produced by the determined allelic profile, wherein increased risk of developing breast cancer is associated with increased production of 4-OHE₂ and/or E₂-3,4-Q as compared to mean production by a relevant genetic population, and reduced risk of developing breast cancer is associated with reduced production of 4-OHE₂ and/or E₂-3,4-Q as compared to mean production by a relevant genetic population. Increased/decreased risk may be associated with increased/decreased production of E₂-3,4-Q, or 4-OHE₂ individually, or both 4-OHE₂ and E₂-3,4-Q. The sample is derived from oral tissue or blood.

The model may adjust the relative ratio of CYP1B1/CYP1A1, for example, to account for environmental influences. Such adjusted ratio may be 2:1, 3:1, 4:1, 5:1, 6:1 or 10:1. The method may further comprise assessing one or more aspects of the subject's personal history, such as age, ethnicity, reproductive history, menstruation history, use of oral contraceptives, body mass index, alcohol consumption history, smoking history, exercise history, diet, family history of breast cancer or other cancer including the age of the relative at the time of their cancer diagnosis, and a personal history of breast cancer, breast biopsy or DCIS, LCIS, or atypical hyperplasia.

Determining the allelic profile may be achieved by amplification of nucleic acid from the sample, such as by PCR. Primers for such amplification may be located on a chip, and may be specific for alleles of the genes. The method may also further comprise cleaving amplified nucleic acid. The method may also further comprise making a decision on the timing and/or frequency of cancer diagnostic testing for the subject and/or making a decision on the timing and/or frequency of prophylactic cancer treatment for the subject.

In another embodiment, there is provided a method for determining the need for routine diagnostic testing of a female subject for breast cancer comprising (a) determining, in a sample from the subject, the allelic profile of COMT, CYP1A1 and CYP1B1; and (b) predicting, based an in silico model of estrogen biosynthesis, relative amounts of 4-OHE₂ and/or E₂-3,4-Q produced by the determined allelic profile, wherein need for routine diagnostic testing is associated with increased production of 4-OHE₂ and/or E₂-3,4-Q as compared to mean production by a relevant genetic population. The need for routine testing may be associated with increased production of E₂-3,4-Q or 4-OHE₂ individually, or both 4-OHE₂ and E₂-3,4-Q.

In still yet another embodiment, there is provided a method for determining the need of a female subject for prophylactic anti-breast cancer therapy comprising (a) determining, in a sample from the subject, the allelic profile of COMT, CYP1A1 and CYP1B1; and (b) predicting, based an in silico model of estrogen biosynthesis, relative amounts of 4-OHE₂ and/or E₂-3,4-Q produced by the determined allelic profile, wherein need for prophylactic breast cancer therapy is associated with increased production of 4-OHE₂ and/or E₂-3,4-Q as compared to mean production by a relevant genetic population. The need for prophylactic breast cancer therapy may be associated with increased production of E₂-3,4-Q or 4-OHE₂ individually, or both 4-OHE₂ and E₂-3,4-Q.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—The estrogen metabolism pathway is regulated by oxidizing phase I and conjugating phase II enzymes. CYP1A1 and CYP1B1 catalyze the oxidation of E₂ to catechol estrogens 2-OHE₂ and 4-OHE₂. The catechol estrogens are either methylated by COMT to methoxyestrogens (2-MeOE₂, 2-OH-3-MeOE₂, 4-MeOE₂) or further oxidized by CYPs to semiquinones (E₂-2,3-SQ, E₂-3,4-SQ) and quinones (E₂-2,3-Q, E₂-3,4-Q). The estrogen quinones are conjugated by GSTP1 to GSH-conjugates (2-OHE₂-1-SG, 2-OHE₂-4-SG, 4-OHE₂-2-SG).

FIGS. 2A-B—(FIG. 2A) Comparison of mathematical model with experimental data. The metabolism of E₂, 2-OHE₂, 4-OHE₂, 2-MeOE₂, 2-OH-3-MeOE₂, 4-MeOE₂, 2-OHE₂-1-SG, 2-OHE₂-4-SG, and 4-OHE₂-2-SG is shown as a function of time. In each graph, the concentration is expressed in μM, the blue dots represent the experimental data (Dawling et al., 2004), and the red curves are derived from the mathematical model. (FIG. 2B) Simulated kinetics of estrogen quinones E₂-2,3-Q and E₂-3,4-Q.

FIGS. 3A-D—Kinetic-genomic modeling of catechol estrogens. (FIG. 3A) 2-OHE₂, (FIG. 3B) 4-OHE₂ and estrogen quinones (FIG. 3C) E₂-2,3-Q and (FIG. 3D) E₂-3,4-Q were modeled using rate constants for wild-type and variant CYP1A1, CYP1B1, and COMT. Only the highest, lowest, and wild-type (dotted line) AUCs are shown.

FIGS. 4A-C—Correlation of E₂-3,4-Q AUC with CYP1B1/CYP1A1 ratio for cases and controls. (FIG. 4A) Box and whisker graph of E₂-3,4-Q AUCs for entire population of 221 cases (red) and 217 controls (blue). Each box includes 84% of the respective group while the whiskers represent the top and bottom 8 percentiles. As indicated by the medians (center line in each box), the AUCs for cases and controls rise with increasing CYP ratio. However, there are no significant differences between case and control medians at any CYP ratio tested (see p-values). (FIG. 4B) Column scatter graph of E₂-3,4-Q AUCs for top 8 percentile (35 subjects) of entire study population. Each dot represents an individual case (red) or control (blue). Subjects with the same composite CYP1A1-CYP1B1-COMT enzyme haplotype have the same E₂-3,4-Q AUC. As the CYP ratio increases, their E₂-3,4-Q AUC changes in the same manner. However, subjects with different composite enzyme haplotypes may yield different E₂-3,4-Q AUC values, resulting in a change in their ranking with increasing CYP ratio. (FIG. 4C) Column scatter graph of E₂-3,4-Q AUCs for top 2 percentile (10 subjects) of entire study population. There are significantly more cases (red) than controls (blue) (p=0.01 at CYP1B1/CYP1A1=5).

### DESCRIPTION OF ILLUSTRATIVE EMBODIMENTS

Despite considerable progress in cancer therapy, cancer mortality rates continue to be high. Generally, the poor prognosis of many cancer patients derives from the failure to identify the disease at an early stage, i.e., before metastasis has occurred. While not trivial, treatment of organ confined primary tumors is far more likely to be successful than any treatment for advanced, disseminated malignancies.

In order to affect early diagnosis of cancer, at a time when patients still appear healthy, it is necessary to screen large numbers of individuals. However, the costs associated with such testing, and the unnecessary follow-ups occasioned by false positive results, are prohibitive. Thus, it is necessary to find better ways of assessing cancer risk in the general population and concentrating preventative and early detection efforts on those individuals at highest risk.

### I. THE PRESENT INVENTION

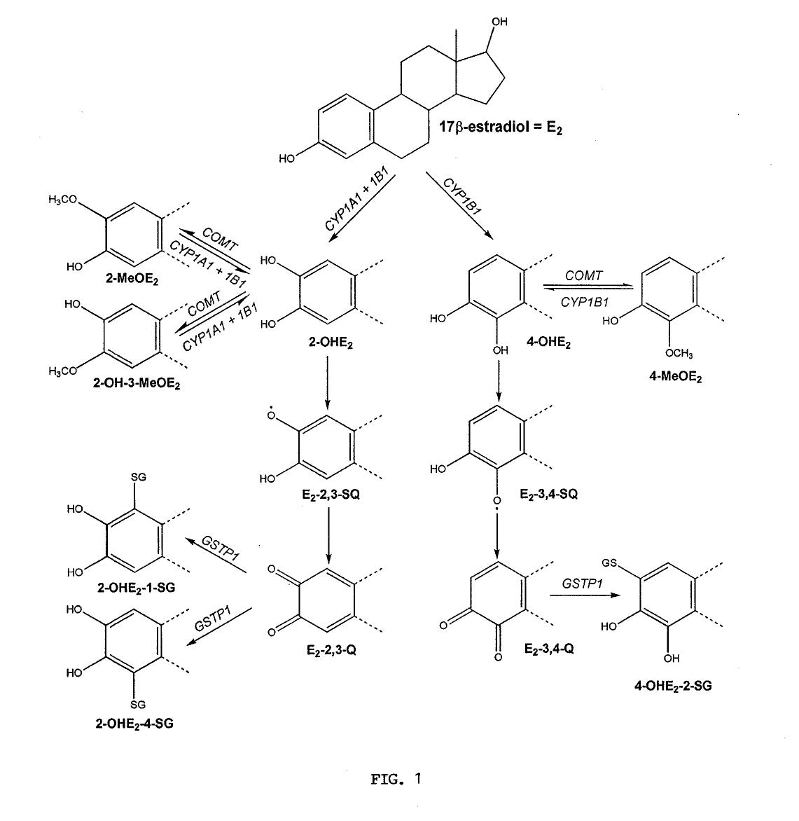

Numerous epidemiological studies have implicated estrogens in the development of breast cancer (Brown et al., 1976; Chen et al., 1983). The two major estrogens, E₂ and estrone (E₁), are ligands for the estrogen receptor and substrates for oxidizing phase I enzymes, CYP1A1 and CYP1B1. In their dual role of ligand and substrate, estrogens may simultaneously stimulate cell proliferation and gene expression via the estrogen receptor and cause DNA damage via their oxidation products, the catechol estrogens (Corchero et al., 2001; Gorman et al., 1983). The latter mechanism is based on the unique chemical structure of estrogens. Unlike all other steroid hormones, estrogens have an aromatic A-ring, which yields catechols upon oxidation that may be further oxidized to highly reactive semiquinones and quinones (FIG. 1), which, in turn, can form both oxidative and estrogen DNA adducts. Thus, estrogen quinones appear to share a common feature of many chemical carcinogens, i.e., the ability to covalently modify DNA (Gorman, 1993; Hildebrand et al., 1985a; Hildebrand et al., 1985b; Jaiswal et al., 1987). Support for the carcinogenic activity of estrogens and their oxidative products, the catechol estrogens, comes from experiments in animal models. Treatment with E₂ and the catechol 4- and 2-hydroxyestrogens caused kidney cancer in male Syrian hamsters and endometrial cancer in female CD1 mice (Jaiswal et al., 1985a; Jaiswal et al., 1985b; Jaiswal and Nebert, 1986). However, there is no animal model for estrogen-induced breast cancer and even in the hamster and mouse models the precise mechanism of DNA damage is uncertain. Thus, there is a need to understand estrogen metabolism in the human breast in order to elucidate the role of endogenous and exogenous estrogens in mammary carcinogenesis. To advance this understanding requires not only characterization of the various estrogen metabolites but equally important a precise definition of the responsible enzymes.

Several investigators have proposed a qualitative model of mammary estrogen metabolism regulated by oxidizing phase I and conjugating phase II enzymes (Jaiswal et al., 1986; Jaiswal et al., 1987). The oxidative estrogen metabolism pathway starts with E₂ and E₁, which are oxidized to the 2-OH and 4-OH catechol estrogens by the phase I enzymes CYP1A1 and CYP1B1 (Jiang et al., 2005; Jones et al., 1991). These same enzymes are postulated to further oxidize the catechol estrogens to unstable semiquinones and quinones. Estrogen quinones then form Michael addition products with deoxynucleosides (Gorman, 1993; Hildebrand et al., 1985; Kawajiri et al., 1996). The catechol estrogens and their estrogen quinones/semiquinones also undergo redox cycling, which results in the production of reactive oxygen species capable of causing oxidative DNA damage (Kawajiri et al., 1990; Kawajiri et al., 1986; Kouri et al., 1982). Thus, P450-mediated estrogen metabolism is expected to lead to the formation of both oxidative and estrogen DNA adducts, all of which have been shown to possess mutagenic potential (McBride, 1985; Mooney et al., 1997; Nakachi et al., 1991). It is postulated that the genotoxicity of the oxidative estrogen metabolism pathway is mitigated by alternate reactions of the metabolites with phase II enzymes. Specifically, COMT catalyzes the methylation of catechol estrogens to methoxy estrogens, which lowers the catechol estrogens available for conversion to estrogen quinones (Nebert, 1988; Nebert and Gonzalez, 1987). In turn, the estrogen quinones undergo conjugation with GSH via the catalytic action of GSTP1 (Ocraft et al., 1985; Paolini et al., 1999). The formation of GSH-estrogen conjugates would reduce the level of estrogen quinones and thereby lower the potential for DNA damage.

The current model of mammary estrogen metabolism has several limitations. First, only single enzymes, e.g., CYP1B1 and COMT, have been analyzed to date with simple substrate-product kinetics, which clearly generates an incomplete picture of the metabolic pathway. Second, while the model incorporates the functional roles of the phase I and II enzymes, it remains uncertain how the enzymes interact quantitatively. Third, each of the phase I and II enzymes contains genetic polymorphisms (Jones et al., 1991; Nebert, 1988; Perera, 1997; Petersen et al., 1991). Studies from several laboratories, including our own, have examined the functional implications of the polymorphisms on estrogen metabolism, again focusing on single enzymes (Jones et al., 1991; Nebert, 1988; Nebert and Gonzalez, 1987; Quattrochi et al., 1985; Thum and Borlak, 2000). Thus, the multitude of potential kinetic reactions resulting from the complex genetic variations of the phase I and II enzymes is completely outside the scope of the current model of estrogen metabolism.

The inventor recently developed an experimental in vitro model of mammary estrogen metabolism, in which he used purified, recombinant phase I enzymes CYP1A1 and CYP1B1 with the phase II enzymes COMT and GSTP1 to determine how E₂ is metabolized (Dawling et al., 2004). Both gas and liquid chromatography were employed with mass spectrometry (GC/MS and LC/MS) to measure the parent hormone E₂ as well as eight metabolites, i.e., the catechol estrogens, methoxyestrogens, and estrogen-GSH conjugates (FIG. 1). Here, the inventor used these experimental data to develop a multi-compartmental kinetic model of the metabolic pathway. Furthermore, previously determined rate constants of variant CYP1A1, CYP1B1, and COMT were used to present an in silico kinetic-genomic model of mammary estrogen metabolism. Finally, as discussed more fully in the Examples, the model was applied to a breast cancer case-control population and it was determined that the combination of enzyme haplotypes with elevated 4-OHE₂ and/or E₂-3,4-Q production did indeed identify a subset of women with increased breast cancer risk.

### II. ENZYMES AND ENZYMATIC PATHWAYS OF ESTROGEN METABOLISM

Glutathione S-transferases (GSTs) constitute a superfamily of ubiquitous, multifunctional enzymes, which play a key role in cellular detoxification (Strange et al., 2001). The GSTs catalyze the conjugation of the tripeptide glutathione (GSH) to a wide variety of exogenous and endogenous chemicals with electrophilic functional groups (e.g., products of oxidative stress, environmental pollutants, and carcinogens), thereby neutralizing their electrophilic sites, and rendering the products more water-soluble (Hayes et al., 1995). Based on sequence homology and immunological crossreactivity, human cytosolic GSTs have been grouped into seven families, designated GSTα, Mu, Pi, Sigma, Omega, Theta, and Zeta (Elston et al., 1977; Mannervik et al., 1992). The GSTα subfamily is encoded by a 100-kb gene cluster at 1p13.3 arranged as 5′-GSTM4-GSTM2-GSTM1-GSTM5-GSTM3-3′ (Xu et al., 1998). Deletion of the GSTM1 gene, GSTM1-0 frequently affects both alleles, resulting in the so-called null genotype, GSTM1−/−. A meta-analysis of 30 studies involving over 10,000 individuals identified the GSTM1 null genotype in 53% Caucasians, with a 42 to 60% range for individual studies (Bailey et al., 1998; Garte et al., 2001). The frequency of the GSTM1 null genotype was similar in Asians but lower in African-Americans, 27% (16-36%). Detailed mapping of the GSTα gene cluster revealed that the GSTM1 gene is flanked by two almost identical 4.2-kb regions. The GSTM1-0 deletion is caused by homologous recombination involving the left and right 4.2-kb repeats (Xu et al., 1998). Analysis of 20 GSTM1-0 alleles from 13 unrelated individuals showed the same recombination pattern which results in a 16-kb deletion containing the entire GSTM1 gene. The GSTM1 gene is excised relatively precisely leaving the adjacent GSTM2 and GSTM5 genes intact. Therefore, one can rule out recombination with neighboring GSTM genes as a possible mechanism for the GSTM1-0 deletion, despite extensive homologies in certain regions.

In view of the importance of GSTs in cellular detoxification, the enzyme deficiency associated with the GSTM1 null genotype has attracted considerable attention with regard to cancer epidemiology. A search of the literature published from 1993 to 2003 listed over 500 studies of the GSTM1 genotype in relation to lung, breast, colon, brain, and various other types of cancer. These studies have in common PCR-based genotyping using an assay designed to identify the wild-type (wt) allele of GSTM1 (Rebbeck, 1997). The absence of a PCR product (273 bp) indicates the GSTM1 null genotype. Consequently, study participants were categorized as either wt or null “genotypes.” This analytical approach has one basic flaw in that it does not positively identify the null allele and, therefore, cannot distinguish homozygous wt/wt from heterozygous wt/-individuals. Assuming that the presence of 2, 1, or 0 GSTM1 alleles is associated with a gene dosage effect resulting in high-, low-, or non-GSTM1 conjugator phenotypes, the current approach oversimplifies phenotypes as all or none. Not surprisingly, the large number of studies utilizing this approach has yielded confusing data, which resulted in inconsistent or contradictory publications on the association of the GSTM1 “genotype” with various malignancies (Dunning et al., 1999; Geisler et al., 2001; Seidegard et al., 1988).

Estrogens are clearly carcinogenic in humans and rodents but the molecular pathways by which these hormones induce cancer are only partially understood. In broad terms, two distinct mechanisms of estrogen carcinogenicity have been outlined. Stimulation of cell proliferation and gene expression by binding to the estrogen receptor is one important mechanism in hormonal carcinogenesis (Nandi et al., 1995). However, estrogenicity is not sufficient to explain the carcinogenic activity of all estrogens because some estrogens are not carcinogenic. Increasing evidence of a second mechanism of carcinogenicity has focused attention on catechol estrogen metabolites, which are less potent estrogens than 17β-estradiol (E2), but can directly or indirectly induce various types of DNA damage ranging from modification of bases to single-strand breakage, all of which are thought to have mutagenic potential (Cavalieri et al., 1997; Floyd et al., 1990; Han et al., 1994; Yager et al., 1996).

The two main estrogens, E2 and estrone (E1), are metabolized to catechol estrogens, their 2-OH and 4-OH derivatives. Two phase I enzymes, CYP1A1 and CYP1B1, are responsible for the hydroxylation of E2 and E1 to the 2-OH and 4-OH catechol estrogens (i.e., 2-OHE1,2-OHE2,4-OHE1, and 4-OHE2;). The 2-OH and 4-OH catechol estrogens are oxidized to semiquinones (E1-2,3SQ, E2-2,3SQ, E1-3,4SQ, and E2-3,4SQ) and quinones (E1-2,3Q, E2-2,3Q, E1-3,4Q, and E2-3,4Q). The latter are highly reactive electrophilic metabolites that are capable of forming DNA adducts (Abul-Hajj et al., 1988; Dwivedy et al., 1992). Further DNA damage results from quinone-semiquinone redox cycling, generated by enzymatic reduction of catechol estrogen quinones to semiquinones and subsequent autoxidation back to quinones (Liehr et al., 1986; 1990; Liehr, 1990). Two phase II enzymes, i.e., catechol-O-methyltransferase (COMT) and glutathione S-transferases (GSTs), either inactivate catechol estrogens or protect against estrogen carcinogenesis by detoxifying products of oxidative damage that may arise upon redox cycling of catechol estrogens. COMT inactivates 2-OH and 4-OH catechol estrogens by O-methylation, forming 2-MeO and 4-MeO methoxy estrogens (Roy et al., 1990). GSTP1, and GSTT1 inactivate catechol estrogen quinones by conjugation with glutathione (Iverson et al., 1996).

Although other cytochrome P450 enzymes, such as CYP1A2 and CYP3A4, are involved in hepatic and extrahepatic estrogen hydroxylation, CYP1A1 and CYP1B1 display the highest level of expression in breast tissue (Huang et al., 1997; Shimada et al., 1996). In turn, CYP1B1 exceeds CYP1A1 in its catalytic efficiency as E2 hydroxylase and differs from CYP1A1 in its principal site of action (Hayes et al., 1996; Spink et al., 1992; Spink et al., 1994). CYP1B1 has its primary activity at the C-4 position of E2, whereas CYP1A1 has its primary activity at the C-2 position in preference to 4-hydroxylation. Thus, CYP1B1 appears to be the main cytochrome P450 responsible for the 4-hydroxylation of E2. The 4-hydroxylation activity of CYP1B1 has received particular attention due to the fact that the 2-OH and 4-OH catechol estrogens differ in carcinogenicity. Treatment with 4-OHE2 and 4-OHE1, but not 2-OHE2 and 2-OHE1, induced renal cancer in Syrian hamster (Li et al., 1987; Liehr et al., 1986). Analysis of renal DNA demonstrated that 4-OHE2 and 4-OHE1 significantly increased levels of the oxidized base 8-hydroxy-deoxyguanosine, while 2-OHE2 did not cause oxidative DNA damage (Han et al., 1995). Similarly, 4-OHE2 induced DNA single-strand breaks while 2-OHE2 had a negligible effect. Comparison of the corresponding catechol estrogen quinones showed that E2-3,4Q and E1-3,4Q produced two to three orders of magnitude higher levels of depurinating DNA adducts than E2-2,3Q and E1-2,3Q (Cavalieri et al., 1997). Finally, examination of microsomal E2 hydroxylation in human breast cancer showed significantly higher 4-OHE2/2-OHE2 ratios in tumor tissue than in adjacent normal breast tissue (Liehr et al., 1996). All these findings support a causative role of 4-OH catechol estrogens in carcinogenesis and implicate CYP1B1 as a key player in the process.

Genetic variants of each of the enzymes involved in catechol estrogen metabolism have been identified. The CYP1A1 gene possesses four polymorphisms of which two result in amino acid substitutions: codon 461Thr→Asn and codon 462Ile→Val (Cascorbi et al., 1996; Hayashi et al., 1991). Six polymorphisms of the CYP1B1 gene have been described, of which four result in amino acid substitutions (Bailey et al., 1998; Stoilov et al., 1998). Two of these amino acid substitutions: codon 432Val→Leu and codon 453Asn→Ser) have been described (Bailey et al., 1998). Stoilov et al. (1998) described the other two amino acid substitutions in codons 48 (Arg→Gly) and 119 (Ala→Ser). The COMT gene possesses a common polymorphism in codon 158Val→Met (Lachman et al., 1996). The GSTP1 gene contains polymorphisms in codons 105Ile→Val and 113Ala→Val (Ali-Osman et al., 1997; Zimniak et al., 1994). The functional implications of these polymorphisms in terms of enzyme activities have been investigated. The 462Ile→Val substitution in recombinant variant CYP1A1 does not appear to alter enzymatic activity (Persson et al., 1997; Zhang et al., 1996). However, in vivo CYP1A1 activity was more readily inducible in lymphocytes with the Val/Val genotype than in wild type lymphocytes (Cosma et al., 1993). Recombinant wild-type and each of the polymorphic variants of CYP1B1 were expressed and purified, followed by assays of E2 hydroxylation activity (Hanna et al., 2000). Quantitation of 2-OH-E2 and 4-OH-E2 by gas chromatography/mass spectrometry showed that the CYP1B1 variants displayed 2.4- to 3.4-fold higher catalytic efficiencies than the wild type enzyme. Using catecholamines as substrate, Syvanen et al. (1997) determined that COMT activity in red blood cells from individuals with the homozygous Met/Met genotype was reduced two-thirds compared to individuals with the homozygous Val/Val wild-type. Heterozygotes showed intermediate activity. It is likely that the polymorphism in codon 158Val→Met affects O-methylation of catechol estrogens in a similar manner because both catecholamines and catechol estrogens are recognized as catechol substrates by COMT. The GSTP1 polymorphisms in codons 105Ile→Val and 113Ala→Val are associated with a 3- to 4-fold reduction in catalytic activity compared to wild-type GSTP1 (Ali-Osman et al., 1997; Zimniak et al., 1994). Approximately 20% of individuals possess the homozygous null GSTT1 genotype and are therefore devoid of functional GSTT1 enzyme (Wiencke et al., 1995). Thus, inherited alterations in the activity of any of these six enzymes may be associated with significant changes in estrogen metabolism. The associated interindividual differences in life-long exposure to carcinogenic catechol estrogens hold the potential to explain differences in breast cancer risk.

The inventor previously described the association of various alleles of CYP1A1, CYP1B1 and COMT in U.S. Patent Publication No. 2005/0255504, the entire contents of which are hereby incorporated by reference. Therein, methods for identifying a subject having an increased risk of developing cancer, for example, breast cancer, are provided comprising determining the presence of the homozygous wild-type genotype of GSTM1s, wherein the presence of the homozygous wild-type genotype of GSTM1s identifies a subject with increased risk of cancer. Also provided were methods for identifying a subject having a reduced risk of cancer, for example, breast cancer, comprising determining the presence of the homozygous null allele genotype of GSTM1s, wherein the presence of the homozygous null allele genotype of GSTM1s identifies a subject with decreased risk of cancer.

A. CYP1A1

CYP1A1 (Accession No. NM 000499) is predicted to have 512 amino acids and has an estimated molecular weight of 58,165 daltons. Cytochrome P1-450 is the form of P-450 most closely associated with polycyclic-hydrocarbon-induced AHH activity. Chen et al. (1983) cloned a portion of the genomic gene. The compound 2,3,7,8-tetrachlorodibenzo-p-dioxin (TCDD) is a potent inducer of many proteins including drug-metabolizing enzymes such as the cytochrome P-450 proteins. The P1-450 that is induced by TCDD is the same as AHH. Jaiswal et al. (1985) used a human cell line in which treatment with TCDD resulted in high levels of AHH(P1-450) activity and of human P1-450. Jaiswal et al. (1985) estimated that the TCDD-inducible P-450 gene family diverged from the phenobarbital-inducible P-450 gene family (see 122720) more than 200 million years ago. Nebert and Gonzalez (1987) estimated that this divergence occurred more than 750 million years ago. Kouri et al. (1982) reported that individuals with the high-inducibility phenotype (present in approximately 10% of the human population) might be at greater risk than low-inducibility individuals for cigarette smoke-induced bronchogenic carcinoma. In a 3-generation family of 15 individuals, Petersen et al. (1991) showed that the high-CYP1A1-inducibility phenotype segregated concordantly with an infrequent polymorphic site located 450 bases downstream from the CYP1A1 gene. These findings were consistent with those of Kawajiri et al. (1986; 1990), who demonstrated an association between this polymorphism and an increased incidence of squamous cell lung cancer.

Quattrochi et al. (1985) cloned human P450DX genes and concluded that there are at least 2 in humans. Hildebrand et al. (1985) used a full-length cDNA for human 2,3,7,8-tetrachlorodibenzo-p-dioxin (TCDD)-inducible cytochrome P1-450 to study DNA from somatic hybrid cells. They assigned the gene to chromosome 15. Jaiswal and Nebert (1986) indicated that this locus is in the 15q22-qter segment, near MPI. The P3-450 gene (CYP1A2) has also been located on chromosome 15.

Corchero et al. (2001) found that the CYP1A1 and CYP1A2 genes are separated by a 23-kb segment that contains no other open reading frames. They are in opposite orientation, revealing that they share a common 5-prime flanking region. Analysis of the sequence demonstrated the presence of xenobiotic response elements (XREs) previously reported for CYP1A1 and CYP1A2 and several additional consensus sequences for putative XREs. The presence of all the XREs upstream of both genes suggested that some of the regulatory elements known to control CYP1A1 gene expression could also control CYP1A2 gene expression.

The nomenclature and symbolization of the P450 enzymes and their genes have gone through many changes. The currently preferred system (Nebert, 1988) uses the symbol CYP followed by a number for family and a letter for subfamily. CYP1 is the designation of the family of P450 genes located on human chromosome 15 and mouse chromosome 9. (CYP1 was previously used for a P450 gene on chromosome 19, which is now called CYP2.) The number assigned to the family is sometimes arbitrary or selected for reasons of historical priority; in other cases it has specific significance, e.g., in the case of CYP21 on 6p and CYP17 on 10, which are genes for the enzymes of classes designated P450XXI (steroid 21-hydroxylase) and P450XVII (steroid 17-α-hydroxylase), respectively.

Hildebrand et al. (1985) showed that in the mouse, which has 2 dioxin-inducible P-450 genes, P1-450 and P3-450, the 2 genes are situated in the middle portion of chromosome 9 near the Mpi-1 locus, between Thy-1 and Pk-3. Treatment of mice with polycyclic aromatic hydrocarbons results in induction of P1-450 and P3-450. Their genes have been cloned and shown to be coordinately regulated by the cytosolic receptor which is coded by the Ah locus and specifically binds the inducing chemicals.

By Southern blot analysis of DNA from hamster-mouse somatic cell hybrids, Tukey et al. (1984) demonstrated that the genes for P1-450 and P3-450 map to chromosome 9 in the mouse. The major regulatory gene controlling P1-450 induction in the mouse is located in the centromeric region of chromosome 12. Mouse chromosome 9 shows other homology of synteny with human 15. Jaiswal et al. (1985) and Kawajiri et al. (1986) isolated and analyzed the complete nucleotide sequence of a human genomic clone highly homologous to the rat cytochrome P-450 that is induced by methylcholanthrene and TCDD. A fusion gene, which was constructed by ligating the 5-prime flanking region of the gene to the structural gene for prokaryotic chloramphenicol acetyltransferase (CAT), expressed the CAT activity in mouse cells in response to administered methylcholanthrene. Thus, the isolated human gene was indeed one for methylcholanthrene inducibility.

Polycyclic aromatic hydrocarbons (PAHs) generated from the combustion of fossil fuels, and aromatic amines, which are present in cigarette smoke and other environmental media, present 2 classic environmental carcinogens. Perera (1997) reviewed evidence on variation and susceptibility to the effects of these carcinogens. CYP1A1 encodes a phase I cytochrome P450 enzyme that metabolizes PAHs such as benzo[a]pyrene (BP). About 10% of Caucasians have a highly inducible form of the enzyme that is associated with an increased risk of lung cancer in smokers. Although not all studies have been positive, in Japanese and certain Caucasian populations, increased lung cancer risk was correlated with 1 or both CYP1A1 polymorphisms: the so called MSPI polymorphism and the closely-linked exon 7 (isoleucine-valine) polymorphism (Kawajiri et al., 1996; Nakachi et al., 1991). The greatest incremental lung cancer risk from the “susceptible” CYP1A1 genotype was seen in light smokers (7 times the risk of light smokers without the genotype), whereas heavy smokers with this genotype had less than twice the risk of heavy smokers without the genotype. The proposed mechanism for the increased risk is higher CYP1A1 inducibility or enhanced catalytic activity of the valine-type CYP1A1 enzyme. Consistent with these mechanisms, Mooney et al. (1997) found that U.S. smoking volunteers with the exon 7 mutation had more PAH-DNA adducts in their white blood cells than did smokers without the variant. Perera (1997) stated that PAH-DNA adducts were also elevated in cord blood and placenta of newborns with the CYP1A1 MSP1 polymorphism, which suggested that the genetic polymorphism may increase risk from transplacental PAH exposure. In lung tissue of adults, adduct concentration correlated with CYP1A1 expression or enzyme activity. Perera (1997) noted that lung tumors of Japanese smokers were found to be significantly more likely to have p53 mutations if they had the susceptible CYP1A1 genotype. A failure to demonstrate genetic susceptibility through CYP1A1 polymorphism when exposure to the environmental carcinogen is heavy is observed with some other polymorphisms and carcinogenic exposures. It is possible that at higher exposures, the effects of the genetic traits are overwhelmed by the environmental insults.

Numerous studies have shown that maternal cigarette smoking during pregnancy is associated with reduced birth weight and increased risk of low birth weight, defined as weight less than 2,500 g. Maternal cigarette smoking has thus been identified as the single largest modifiable risk factor for intrauterine growth restriction in developed countries. However, not all women who smoke cigarettes during pregnancy have low-birth weight infants. Wang et al. (2002) studied whether the association between maternal cigarette smoking and infant birth weight differs by polymorphisms of 2 maternal metabolic genes: CYP1A1 and GSTT1. The CYP1A1 polymorphism was the Msp1 polymorphism (AA vs Aa and aa); the GSTT1 polymorphism was present versus absent. Wang et al. (2002) found that regardless of genotype, continuous maternal smoking during pregnancy was associated with a mean reduction of 377 g in birth weight. They found that for the CYP1A1 genotype, the estimated reduction in birth weight was 252 g for the AA genotype group, but was 520 g for the Aa/aa genotype group. For the GSTT1 genotype, they found the estimated reduction in birth weight was 285 g and 642 g for the present and absent genotype groups, respectively. When both CYP1A1 and GSTT1 genotypes were considered, Wang et al. (2002) found the greatest reduction in birth weight among smoking mothers with the CYP1A1 Aa/aa and GSTT1 absent genotypes. Among mothers who had not smoked during their pregnancy or during the 3 months prior to their pregnancy, genotype did not independently confer an adverse effect.

The CYP1A1 and CYP1A2 genes are oriented head-to-head on human chromosome 15; the 23.3-kb spacer region might contain distinct regulatory regions for one or the other of these genes, or the regulatory regions for the 2 genes may overlap one another. From 24 unrelated subjects of 5 major, geographically isolated subgroups, Jiang et al. (2005) resequenced both genes (all exons and all introns) plus some 3-prime flanking sequences and the entire spacer region (39.6 kb total). They identified 85 SNPs, 49 of which were not in the NCBI database. SNP typing in 94 Africans, 96 Asians, and 83 Caucasians demonstrated striking ethnic differences in SNP frequencies and haplotype evolution. To demonstrate functionality, they generated a ‘humanized’ BAC transgenic mouse line, having an absence of the mouse orthologous Cyp1a1 or Cyp1a2 genes, that expressed human CYP1A1 and CYP1A2 mRNA, protein, and enzyme activity in a tissue-specific manner similar to that of the mouse.

B. CYP1B1

TCDD (2,3,7,8-tetrachlorodibenzo-p-dioxin), or dioxin, is a prototype for a large class of halogenated aromatic hydrocarbons that are both widespread and persistent chemical pollutants. Sutter et al. (1994) noted that dioxin produces a broad spectrum of toxic responses and is a potent carcinogen and tumor-promoting agent in rodents. In humans, the skin appears to be the most common target organ, and the abnormalities are collectively termed chloracne. Chloracne is characterized by the formation of follicular keratinaceous cysts that may be accompanied by thickening and hyperkeratinization of the interfollicular epidermis. The biologic effects of dioxin are mediated through its high affinity and saturable binding to the dioxin receptor. This receptor is a member of a distinct class of helix-loop-helix transcription factors. As characterized for cytochrome CYP1A1, activation of transcription of dioxin-inducible genes occurs through the binding of the ligand-occupied dioxin receptor to specific DNA recognition sequences within a dioxin-responsive enhancer, found upstream of the mRNA initiation site.

Sutter et al. (1994) reported the isolation and initial characterization of the complete 5.1-kb cDNA corresponding to a TCDD-responsive cDNA clone from a human keratinocyte cell line. A single open reading frame (ORF) that predicted a protein of 543 amino acid residues was determined by computer-assisted analysis of the cDNA sequence, with a predicted molecular weight of 60,846 daltons. This predicted protein identified a new gene subfamily of cytochrome P450, P4501B1 (CYP1B1; Accession No. NM00104), that was mapped to human chromosome 2 by analysis of 2 human/rodent somatic cell hybrid panels. CYP1B1 belongs to a multigene superfamily of monomeric mixed-function monooxygenases, responsible for the phase 1 metabolism of a wide range of structurally diverse substrates by inserting 1 atom of atmospheric oxygen into the substrate molecule, thereby creating a new functional group (e.g., —OH, —NH₂, —COOH). Southern blot analysis of genomic DNA indicated that the human CYP1B subfamily is likely to contain only this single gene. Northern blot analysis of RNA isolated from primary cultures of normal human epidermal keratinocytes showed approximately 100-fold increased levels of the CYP1B1 mRNA after 24-hour treatment with TCDD. Low levels of constitutive CYP1B1 mRNA were detected in 15 different human tissue samples. The results of Sutter et al. (1994) indicated that CYP1B1 is expressed in many normal human tissues.

Tang et al. (1996) refined the mapping of the CYP1B1 gene to 2p22-p21 by fluorescence in situ hybridization. They demonstrated that it contains 3 exons and 2 introns. The putative ORF started in the second exon and was 1,629 bp long. Human CYP1B1 differs from its 2 most closely related members of the cytochrome P450 superfamily, CYP1A1 and CYP1A2, in the number of exons (3 versus 7) and chromosomal location (chromosome 2 versus chromosome 15). A single transcription initiation site was identified. Based on nucleotide sequence analysis, the CYP1B1 gene lacks a consensus TATA box in the promoter region and contains 9 TCDD-responsive enhancer core binding motifs located within a 2.5-kb pair of genomic fragments 5-prime-ward of the transcription start site.

In the study of candidate genes identified in the critical region of 2p21 where a major gene for primary congenital glaucoma, GLC3A, had been mapped by linkage studies, Stoilov et al. (1997) found the CYP1B1 gene, which had previously been identified by Sutter et al. (1994). From a determination of the intron/exon junctions of this gene, Stoilov et al. (1997) concluded that the gene contains 3 exons and 2 introns. The entire coding sequence of the genes is contained in exons 2 and 3. This genomic structure agreed with that reported by Tang et al. (1996). Screening for the presence of coding sequence changes in the CYP1B1 gene, Stoilov et al. (1997) identified 3 different truncating mutations: a 13-bp deletion found in 1 consanguineous and 1 non-consanguineous family; a single cytosine insertion observed in another 2 consanguineous families; and a large deletion found in an additional consanguineous family. In addition, a G-to-C transversion at nucleotide 1640 of the CYP1B1 coding sequence was found that caused a Val432→Leu amino acid substitution. This change created an EcoR57 restriction site, thus providing a rapid screening method. Heterozygosity for the Val432→Leu change was found in 51.4% of 70 normal individuals. This amino acid change was not in that part of CYP1B1 that represented conserved sequences, and both valine and leucine are neutral and hydrophobic. Their very similar aliphatic side groups differ by a single —CH₂ group. Therefore, this change appeared to represent a common amino acid polymorphism that is not related to the primary congenital glaucoma phenotype.

Identification of CYP1B1 as the gene affected in primary congenital glaucoma was said by Stoilov et al. (1997) to be the first example in which mutations in a member of the cytochrome P450 superfamily results in a primary developmental defect. The finding was not unexpected, however, as a link between members of this superfamily and the processes of growth and differentiation had been postulated previously. They speculated that CYP1B1 participates in the metabolism of an as-yet-unknown biologically active molecule that is a participant in eye development. Stoilov et al. (1997) demonstrated that a stable protein product is produced in the affected subjects of these families, and that the 3 mutations they described would be expected to result in a product lacking between 189 and 254 amino acids from the C terminus. This segment harbors the invariant cysteine of all known cytochrome P450 amino sequences; in CYP1B1 it is cys470. Schwartzman et al. (1987) implicated a cytochrome-P450-dependent arachidonate metabolite that inhibits Na+/K+-ATPase in the cornea in regulating corneal transparency and aqueous humor secretion. This finding is consistent with the clouding of the cornea and increased intraocular pressure, the 2 major diagnostic criteria for primary congenital glaucoma.

Stoilov et al. (1998) presented a comprehensive sequence analysis of the translated regions of the CYP1B1 gene in 22 primary congenital glaucoma (PCG) families and 100 randomly selected normal individuals. They identified 16 mutations and 6 polymorphisms, illustrating extensive allelic heterogeneity. The positions affected by these changes were evaluated by building a 3-dimensional homology model of the conserved C-terminal half of CYP1B1. These mutations may interfere with heme incorporation by affecting the hinge region and/or the conserved core structures (CCS) that determine the proper folding and heme-binding ability of P450 molecules. In contrast, all polymorphic sites were poorly conserved and located outside the CCS. Northern hybridization analysis showed strong expression of CYP1B1 in the anterior uveal tract, which is involved in secretion of the aqueous humor and in regulation of outflow facility, processes that could contribute to the elevated intraocular pressure characteristic of PCG. The 22 PCG families were from Turkey, the United States, Canada, and the United Kingdom. Onset of an aggressive form of glaucoma occurred at age 0 to 3 years.

Using homozygosity mapping with a DNA pooling strategy in 3 large consanguineous Saudi primary congenital glaucoma families, Bejjani et al. (1998) found tight linkage to 2p21. Formal linkage analysis in 25 Saudi PCG families confirmed both significant linkage to polymorphic markers in this region and incomplete penetrance, but it showed no evidence of genetic heterogeneity. For these 25 families, the maximum combined 2-point LOD score was 15.76 at a recombination fraction of 0.021, with a polymorphic marker D2S177. Sequence analysis of the coding exons for CYP1B1 in these 25 families revealed 3 distinctive mutations that segregated with the phenotype in 24 families. Additional clinical and molecular data on some mildly affected relatives showed variable expressivity of PCG in this population. Thus, genetic and environmental events must modify the effects of CYP1B1 mutations in ocular development. A small number of PCG mutations identified in this Saudi population made both neonatal and population screening attractive public health measures.

Following up on their report of 3 distinct CYP1B1 mutations in 25 Saudi families segregating PCG, Bejjani et al. (2000) analyzed 37 additional families and confirmed the initial finding of decreased penetrance. Mutations and intragenic single-nucleotide polymorphisms (SNPs) were also analyzed by direct sequencing of all CYP1B1 coding exons. Eight distinct mutations were identified; the most common Saudi mutations, G61E, R469W, and D374N, accounted for 72%, 12%, and 7%, respectively, of all the PCG chromosomes. Five additional homozygous mutations (2 deletions and 3 missense mutations) were detected, each in a single family. Affected individuals from 5 families had no CYP1B1 coding mutations, and each family had a unique SNP profile. The identification of 8 distinct mutations in a single gene, on 4 distinct haplotypes, suggested a relatively recent occurrence of multiple mutations in CYP1B1 in Saudi Arabia. In 22 families, 40 apparently unaffected individuals had mutations and haplotypes identical to their affected sibs. Of these, 2 were subsequently diagnosed with glaucoma and 2 others had abnormal ocular findings consistent with milder forms of glaucoma. Analysis of these 22 kindreds suggested the presence of a dominant modifier locus that is not linked genetically to CYP1B1. Linkage and Southern analyses excluded 3 candidate modifier loci, arylhydrocarbon receptor (AHR) on 7p15, the arylhydrocarbon receptor nuclear translocator (ARNT) on 1q21, and the CYP2D6 gene on 22q13.1.

Vincent et al. (2002) stated that “early-onset glaucoma” refers to genetically heterogeneous conditions for which glaucoma manifests at age 5 to 40 years and for which only a small subset had been molecularly characterized. They studied the role of the MYOC, CYP1B1, and PITX2 genes in 60 patients with juvenile or early-onset glaucoma. By a combination of SSCP and direct cycle sequencing, MYOC mutations were detected in 8 (13.3%) and CYP1B1 mutations in 3 (5%); no PITX2 mutations were detected. The range of phenotypic expression associated with MYOC and CYP1B1 mutations was greater than expected. MYOC mutations included cases of juvenile glaucoma with or without pigmentary glaucoma and mixed-mechanism glaucoma. CYP1B1 mutations involved cases of juvenile open angle glaucoma as well as cases of congenital glaucoma. The study of a Canadian family with autosomal dominant glaucoma showed the segregation of both MYOC and CYP1B1 mutations with disease; however, in this family, the mean age at onset of carriers of the MYOC mutation alone was 51 years, whereas carriers of both the MYOC and CYP1B1 mutations had an average age at onset of 27 years. This work emphasized the genetic heterogeneity of juvenile glaucoma, and suggested that it and congenital glaucoma are allelic variants and that the spectrum of expression of MYOC and CYP1B1 mutations is greater than expected. It also appeared that CYP1B1 may act as a modifier of MYOC expression and that these 2 genes may interact through a common pathway.

Vincent et al. (2001) reported compound heterozygosity for a missense mutation and a nonsense mutation in the CYP1B1 gene in a male of Native Indian (Mohawk)/French Canadian background with Peters anomaly with secondary congenital glaucoma. Ming and Muenke (2002) stated that mutations in CYP1B1 are present in a substantial proportion of patients with congenital glaucoma. Both CYP1B1 and the MYOC gene are expressed in the iris, trabecular meshwork, and ciliary body of the eye.

Activation of 17-p-estradiol (E2) through the formation of catechol estrogen metabolites and the C-16-α hydroxylation product has been postulated to be a factor in mammary carcinogenesis. CYP1B1 exceeds other P450 enzymes in both estrogen hydroxylation activity and expression level in breast tissue. To determine whether inherited variants of CYP1B1 differ from wild-type CYP1B1 in estrogen hydroxylase activity, Hanna et al. (2000) expressed recombinant wild-type and 5 polymorphic variants. They found that the activity of variant enzymes exceeded that of wild-type CYP1B1. The authors suggested that interindividual differences in breast cancer risk associated with estrogen-mediated carcinogenicity may be related to these polymorphisms.

A heterozygous Arg368→His mutation in the CYP1B1 gene (R368H) can modify the age at onset of primary open angle glaucoma (POAG) caused by a heterozygous gly399-to-val mutation in the MYOC gene (G399V). Mutation in the CYP1B1 gene is a major cause of primary congenital glaucoma (PCG). Mutation in the CYP1B1 gene has also been associated with cases of juvenile-onset glaucoma in some families in which other members have PCG, suggesting that shared or overlapping mechanisms may predispose to both forms of glaucoma. In 2 families, Melki et al. (2004) described the occurrence of PCG and POAG in members of a single sibship, all of whom were compound heterozygous for mutations in the CYP1B1 gene. Neither family had a mutation of the MYOC gene. To investigate the role of CYP1B1 mutations in POAG predisposition, irrespective of the presence of an MYOC mutation, Melki et al. (2004) studied CYP1B1 coding region variation in 236 unrelated French Caucasian POAG patients. They found 11 (4.6%) who carried a mutation of the CYP1B1 gene and no MYOC mutation. The patients showed juvenile or middle-age onset of disease with a median age at diagnosis of 40 years (range, 13 to 52 years), significantly earlier than in non-carrier patients. Apart from 1, all mutations detected in POAG patients were previously associated with PCG. Melki et al. (2004) concluded that mutation in the CYP1B1 gene represents a significant risk for early-onset POAG and may also modify the glaucoma phenotype in patients who do not carry an MYOC mutation.

C. COMT

Catechol-O-methyltransferase (COMT; 271 amino acids, predicted molecular weight 30,037 daltons; Accession Nos. DQ 893411 and NP 000745) catalyzes the transfer of a methyl group from S-adenosylmethionine to catecholamines, including the neurotransmitters dopamine, epinephrine, and norepinephrine. This O-methylation results in one of the major degradative pathways of the catecholamine transmitters. In addition to its role in the metabolism of endogenous substances, COMT is important in the metabolism of catechol drugs used in the treatment of hypertension, asthma, and Parkinson's disease. In blood COMT is found mainly in erythrocytes; in leukocytes it exhibits low activity. Weinshilboum and Raymond (1977) found bimodality for red cell catechol-O-methyltransferase activity. Of a randomly selected population, 23% had low activity. Segregation analysis of family data suggested that low activity is recessive. Scanlon et al. (1979) found that homozygotes have a thermolabile enzyme. Thus, the site of the low COMT mutation is presumably the structural locus. Levitt and Baron (1981) confirmed the bimodality of human erythrocyte COMT. They further showed thermolability of the enzyme in “low COMT” samples, suggesting a structural alteration in the enzyme. Autosomal codominant inheritance of the gene coding for erythrocyte COMT activity was adduced by Floderus and Wetterberg (1981) and by Weinshilboum and Dunnette (1981). Gershon and Goldin (1981) concluded that codominant inheritance was consistent with the family data. Spielman and Weinshilboum (1981) suggested that the inheritance of red cell COMT is intermediate, or codominant, there being 3 phenotypes corresponding to the 3 genotypes in a 2-allele system. The COMT of persons with low enzyme activity is more thermolabile than that of persons with high activity.

Wilson et al. (1984) excluded tight and close linkage of COMT with 21 and 15 loci, respectively. A LOD score of 1.27 at theta=0.1 was found between COMT and phosphogluconate dehydrogenase (PGD), which is on chromosome 1. Gustavson et al. (1973, 1982) reported that COMT activity was about 40% higher in Down syndrome children than in normal controls. They attributed this to dosage effect owing to the location of the COMT gene on chromosome 21. Brahe et al. (1986) studied the expression of human COMT in interspecies somatic cell hybrids and found 27% discordance between human chromosome 21 and human COMT. In further studies of mouse-human cell hybrids with a method permitting direct detection of COMT isozymes in autoradiozymograms, Brahe et al. (1986) located the COMT gene on human chromosome 22. By study of DNAs from a panel of human-hamster somatic cell hybrid lines, Grossman et al. (1991; 1992) mapped COMT to 22q11.1-q11.2. Winqvist et al. (1991) assigned COMT to 22q11.2 by means of Southern blot analysis of somatic cell hybrids and chromosomal in situ hybridization. They concluded that COMT is located proximal to the breakpoint cluster region (BCR) involved in chronic myeloid leukemia (151410). Bucan et al. (1993) mapped the homologous murine gene to chromosome 16, where, as in the human, it is closely linked to the lambda light chain genes.

During experiments aimed at building a contiguous group of YACs spanning 22q11, Dunham et al. (1992) found that the HP500 sequence often deleted in the velocardiofacial syndrome (VCFS) was located within the same 450-kb YAC as the COMT gene. They raised the question of whether low COMT might be responsible for psychotic illness, which is a feature of the VCF syndrome in adolescents and adults (Shprintzen et al., 1992).

Lundstrom et al. (1991) isolated cDNA clones for COMT from a human placenta cDNA library using synthetic oligonucleotides as probes. The clones contained an open reading frame that potentially coded for a 24.4-kD polypeptide, presumably corresponding to the cytoplasmic form of COMT. DNA analysis suggested that the human, as well as the rat, dog, and monkey, has 1 gene for COMT.

Karayiorgou et al. (1997; 1999) found an association between obsessive-compulsive disorder (OCD; 164230) and COMT; the homozygous low activity genotype of the COMT gene was associated with risk for OCD in males. Alsobrook et al. (2002) used a family-based genetic design in haplotype relative risk (HRR) and transmission disequilibrium test (TDT) analyses of the association between OCD and COMT. Fifty-six OCD probands and their parents were genotyped for the COMT locus. Analysis of allele and genotype frequencies between the proband genotypes and the control (parental non-transmitted) genotypes failed to replicate the previous finding of gender divergence and gave no evidence of overall association; furthermore, no linkage was detected by TDT. However, further analysis of the COMT allele frequencies by proband gender gave evidence of a mildly significant association with the low activity COMT allele in female probands (P=0.049), but not in male probands.

The COMT gene is a strong candidate for schizophrenia susceptibility, owing to the role of COMT in dopamine metabolism and the location of the gene within the deleted region in VCFS, a disorder associated with high rates of schizophrenia. Shifman et al. (2002) found a highly significant association between schizophrenia and a COMT haplotype in a large case-control sample in Ashkenazi Jews. In addition to the functional Val158→Met polymorphism, this haplotype included 2 non-coding SNPs at either end of the COMT gene. With this background information, Bray et al. (2003) postulated that the COMT susceptibility haplotype is associated with low COMT expression. To test their hypothesis, they applied quantitative measures of allele-specific expression using mRNA from human brain. They demonstrated that COMT is subject to allelic differences in expression in human brain and that the COMT haplotype implicated in schizophrenia by Shifman et al. (2002) is associated with lower expression of COMT mRNA. They also showed that the 3-prime flanking region SNP that in the study of Shifman et al. (2002) gave greatest evidence for association with schizophrenia is transcribed in human brain and exhibits significant differences in allelic expression, with lower relative expression of the associated allele. They concluded that the haplotype implicated in schizophrenia susceptibility is likely to exert its effect, directly or indirectly, by down-regulating COMT expression.

In 38 populations representing all major regions of the world, Palmatier et al. (2004) studied the frequency of the schizophrenia-associated COMT haplotype reported by Shifman et al. (2002) as well as a 7-site COMT haplotype. Their results supported the relevance of the COMT P2 promoter to schizophrenia. The population data showed that the schizophrenia-associated haplotype varies significantly in frequency around the world and has significant heterogeneity when other markers in COMT are also considered.

Lee et al. (2005) screened for 17 known polymorphisms in the COMT gene in 320 Korean patients with schizophrenia and 379 controls. They identified a positive association of schizophrenia with a non-synonymous SNP at codon 72/22 (membrane/soluble-bound form) causing an ala-to-ser substitution (Ala72→Ser). Lee et al. (2005) showed that the Ala72→Ser substitution was correlated with reduced COMT enzyme activity, and their results supported previous reports that the COMT haplotypes implicated in schizophrenia are associated with low COMT expression.

Frisch et al. (2001) found an association between anorexia nervosa and the COMT Val158 allele (V158→M) in a family-based study of 51 Israeli-Jewish AN trios. Gabrovsek et al. (2004) could not replicate this finding in a combined sample of 372 European AN families, suggesting that the findings of Frisch et al. (2001) were specific to a particular population and that Val158 is in linkage disequilibrium with other molecular variations in the COMT gene, or its vicinity, which were the direct cause of genetic susceptibility to anorexia nervosa. Michaelovsky et al. (2005) studied 85 Israeli-Jewish AN trios, including the original sample of Frisch et al. (2001), comprising 66 anorexia nervosa restricting (AN-R) and 19 binge-eating/purging patients. They performed a family-based TDT analysis for 7 SNPs in the COMT-ARVCF region including the V158→M polymorphism. TDT analysis of 5-SNP haplotypes in the AN-R group revealed overall statistically significant transmission disequilibrium for “haplotype B” (COMT 186C, 408G, 472G [Val158] and ARVCF 659C[pro220] and 524T[val175]) (P less than 0.001), while “haplotype A” (COMT 186T, 408C, 472A[met158] and ARVCF 659T[leu220] and 524C[ala175]) was preferentially not transmitted (P=0.01). Haplotype B was associated with increased risk (RR of 3.38), while haplotype A exhibited a protective effect (RR of 0.40) for AN-R. Preferential transmission of the risk alleles and haplotypes from parents was mostly contributed by fathers.

Sweet et al. (2005) conducted a study to determine if COMT genetic variation was associated with a risk of psychosis in Alzheimer disease (AD). The study included a case-control sample of 373 individuals diagnosed with AD with or without psychosis. Subjects were characterized for alleles at 3 loci previously associated with schizophrenia, rs737865, rs4680, and rs165599, and for a C/T transition adjacent to an estrogen response element (ERE6) in the COMT P2 promoter region. Single-locus and haplotype tests of association were conducted. Logit models were used to examine independent and interacting effects of alleles at the associated loci and all analyses were stratified by sex. In female subjects, rs4680 demonstrated a modest association with AD plus psychosis; rs737865 demonstrated a trend towards an association. There was a highly significant association of AD plus psychosis with a 4-locus haplotype, which resulted from additive effects of alleles at rs4680 and ERE6/rs737865 (the latter were in linkage disequilibrium). In male subjects, no single-locus test was significant, although a strong association between AD with psychosis and the 4-locus haplotype was observed. That association appeared to result from interaction of the ERE6/rs737865, rs4680, rs165599 loci. Genetic variation in COMT was associated with AD plus psychosis and thus appears to contribute to psychosis risk across disorders.

### III. SAMPLE COLLECTION AND PROCESSING

A. Sampling

In order to assess the genetic make-up of an individual, it is necessary to obtain a nucleic acid-containing sample. Suitable tissues include almost any nucleic acid containing tissue, but those most convenient include oral tissue or blood. For those DNA specimens isolated from peripheral blood specimens, blood may be collected in heparinized syringes or other appropriate vessel following venipuncture with a hypodermic needle. Oral tissue may advantageously be obtained from a mouth rinse. Oral tissue or buccal cells may be collected with oral rinses.

B. cDNA Production

In some aspects of the invention, it may be useful to prepare a cDNA population for subsequent analysis. In typical cDNA production, mRNA molecules with poly(A) tails are potential templates and will each produce, when treated with a reverse transcriptase, a cDNA in the form of a single-stranded molecule bound to the mRNA (cDNA:mRNA hybrid). The cDNA is then converted into double-stranded DNA by DNA polymerases such as DNA Pol I (Klenow fragment). Klenow polymerase is used to avoid degradation of the newly synthesized cDNAs. To produce the template for the polymerase, the mRNA must be removed from the cDNA:mRNA hybrid. This is achieved either by boiling or by alkaline treatment (see lecture notes on the properties of nucleic acids). The resulting single-stranded cDNA is used as the template to produce the second DNA strand. As with other polymerases, a double-stranded primer sequence is needed and this is fortuitously provided during the reverse transcriptase synthesis, which produces a short complementary tail at the 5′ end of the cDNA. This tail loops back onto the ss cDNA template (the so-called “hairpin loop”) and provides the primer for the polymerase to start the synthesis of the new DNA strand producing a double stranded cDNA (ds cDNA). A consequence of this method of cDNA synthesis is that the two complementary cDNA strands are covalently joined through the hairpin loop. The hairpin loop is removed by use of a single strand specific nuclease (e.g., S1 nuclease from Aspergillus oryzae).

Kits for cDNA synthesis (SMART RACE cDNA Amplification Kit; Clontech, Palo Alto, Calif.). It also is possible to couple cDNA with PCR™, into what is referred to as RT-PCR™. PCR™ is discussed in greater detail below.

### IV. DETECTION METHODS

Once the sample has been properly processed, detection of sequence variation is required. Perhaps the most direct method is to actually determine the sequence of either genomic DNA or cDNA and compare these to the known alleles. This can be a fairly expensive and time-consuming process. Nevertheless, this is the lead technology of numerous bioinformatics companies with interests in SNPs including such firms as Celera, Curagen, Incyte, Variagenics and Genaissance, and the technology is available to do fairly high volume sequencing of samples. A variation on the direct sequence determination method is the Gene Chip™ method as advanced by Affymetrix. Such chips are discussed in greater detail below.

Alternatively, more clinically robust and less expensive ways of detecting DNA sequence variation are being developed. For example, Perkin Elmer adapted its TAQman™ Assay to detect sequence variation several years ago.

Orchid BioSciences has a method called SNP-IT™ (SNP-Identification Technology) that uses primer extension with labeled nucleotide analogs to determine which nucleotide occurs at the position immediately 3′ of an oligonucleotide probe.

Sequenom uses a hybridization capture technology plus MALDI-TOF (Matrix Assisted Laser Desorption/Ionization-Time-of-Flight mass spectrometry) to detect sequence variation with their MassARRAY™ system.

Promega has the READIT™ SNP/Genotyping System (U.S. Pat. No. 6,159,693). In this method, DNA or RNA probes are hybridized to target nucleic acid sequences. Probes that are complementary to the target sequence at each base are depolymerized with a proprietary mixture of enzymes, while probes which differ from the target at the interrogation position remain intact. The method uses pyrophosphorylation chemistry in combination with luciferase detection to provide a highly sensitive and adaptable SNP scoring system.

Third Wave Technologies has the Invader OS™ method that uses their proprietary Cleavase® enzymes, which recognize and cut only the specific structure formed during the Invader process The Invader OS relies on linear amplification of the signal generated by the Invader process, rather than on exponential amplification of the target. The Invader OS assay does not utilize PCR in any part of the assay.

There are a number of forensic DNA testing labs and many research labs that use gene-specific PCR, followed by restriction endonuclease digestion and gel electrophoresis (or other size separation technology) to detect RFLPs in much the same way that the inventors have. The point is that, how one detects sequence variation (SNPs) is not important in the estimation of cancer risk. The key is the genes and polymorphisms that one examines.

As an alternative SNP detection technology to RFLP, genotypes were determined by Allele Specific Primer Extension (ASPE) coupled to a microsphere-based technical readout. Many accounts of SNP genotyping using microsphere-based methods have been published in the scientific literature. The method is being used as an alternative to RFLP and closely resembles that of Ye et al. (2001). This technology was implemented through the Luminex™-100 microsphere detection platform (Luminex, Austin, Tex.) using oligonucleotide labeled microspheres purchased from MiraiBio, Inc. (Alameda, Calif.).

The following materials and methodologies relate to the present invention, and are therefore described in some detail.

A. Chips

As discussed above, one convenient approach to detecting variation involves the use of nucleic acid arrays placed on chips. This technology has been widely exploited by companies such as Affymetrix, and a large number of patented technologies are available. Specifically contemplated are chip-based DNA technologies such as those described by Hacia et al. (1996) and Shoemaker et al. (1996). These techniques involve quantitative methods for analyzing large numbers of sequences rapidly and accurately. The technology capitalizes on the complementary binding properties of single stranded DNA to screen DNA samples by hybridization. Pease et al. (1994); Fodor et al. (1991).

Basically, a DNA array or gene chip consists of a solid substrate to which an array of single-stranded DNA molecules has been attached. For screening, the chip or array is contacted with a single-stranded DNA sample, which is allowed to hybridize under stringent conditions. The chip or array is then scanned to determine which probes have hybridized. In a particular embodiment of the instant invention, a gene chip or DNA array would comprise probes specific for chromosomal changes evidencing the predisposition towards the development of a neoplastic or preneoplastic phenotype. In the context of this embodiment, such probes could include PCR products amplified from patient DNA synthesized oligonucleotides, cDNA, genomic DNA, yeast artificial chromosomes (YACs), bacterial artificial chromosomes (BACs), chromosomal markers or other constructs a person of ordinary skill would recognize as adequate to demonstrate a genetic change.

A variety of gene chip or DNA array formats are described in the art, for example U.S. Pat. Nos. 5,861,242 and 5,578,832, which are expressly incorporated herein by reference. A means for applying the disclosed methods to the construction of such a chip or array would be clear to one of ordinary skill in the art. In brief, the basic structure of a gene chip or array comprises: (1) an excitation source; (2) an array of probes; (3) a sampling element; (4) a detector; and (5) a signal amplification/treatment system. A chip may also include a support for immobilizing the probe.

In particular embodiments, a target nucleic acid may be tagged or labeled with a substance that emits a detectable signal, for example, luminescence. The target nucleic acid may be immobilized onto the integrated microchip that also supports a phototransducer and related detection circuitry. Alternatively, a gene probe may be immobilized onto a membrane or filter, which is then attached to the microchip or to the detector surface itself. In a further embodiment, the immobilized probe may be tagged or labeled with a substance that emits a detectable or altered signal when combined with the target nucleic acid. The tagged or labeled species may be fluorescent, phosphorescent, or otherwise luminescent, or it may emit Raman energy or it may absorb energy. When the probes selectively bind to a targeted species, a signal is generated that is detected by the chip. The signal may then be processed in several ways, depending on the nature of the signal.

The DNA probes may be directly or indirectly immobilized onto a transducer detection surface to ensure optimal contact and maximum detection. The ability to directly synthesize on or attach polynucleotide probes to solid substrates is well known in the art. See U.S. Pat. Nos. 5,837,832 and 5,837,860, both of which are expressly incorporated by reference. A variety of methods have been utilized to either permanently or removably attach the probes to the substrate. Exemplary methods include: the immobilization of biotinylated nucleic acid molecules to avidin/streptavidin coated supports (Holmstrom, 1993), the direct covalent attachment of short, 5′-phosphorylated primers to chemically modified polystyrene plates (Rasmussen et al., 1991), or the precoating of the polystyrene or glass solid phases with poly-L-Lys or poly L-Lys, Phe, followed by the covalent attachment of either amino- or sulfhydryl-modified oligonucleotides using bi-functional crosslinking reagents (Running et al., 1990; Newton et al., 1993). When immobilized onto a substrate, the probes are stabilized and therefore may be used repeatedly. In general terms, hybridization is performed on an immobilized nucleic acid target or a probe molecule is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), and photopolymers (which contain photoreactive species such as nitrenes, carbenes and ketyl radicals) capable of forming covalent links with target molecules.

Binding of the probe to a selected support may be accomplished by any of several means. For example, DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. DNA may be bound directly to membranes using ultraviolet radiation. With nitrocellose membranes, the DNA probes are spotted onto the membranes. A UV light source (Stratalinker™, Stratagene, La Jolla, Calif.) is used to irradiate DNA spots and induce cross-linking. An alternative method for cross-linking involves baking the spotted membranes at 80° C. for two hours in vacuum.

Specific DNA probes may first be immobilized onto a membrane and then attached to a membrane in contact with a transducer detection surface. This method avoids binding the probe onto the transducer and may be desirable for large-scale production. Membranes particularly suitable for this application include nitrocellulose membrane (e.g., from BioRad, Hercules, Calif.) or polyvinylidene difluoride (PVDF) (BioRad, Hercules, Calif.) or nylon membrane (Zeta-Probe, BioRad) or polystyrene base substrates (DNA.BIND™ Costar, Cambridge, Mass.).

B. Nucleic Acid Amplification Procedures

A useful technique in working with nucleic acids involves amplification. Amplifications are usually template-dependent, meaning that they rely on the existence of a template strand to make additional copies of the template. Primers, short nucleic acids that are capable of priming the synthesis of a nascent nucleic acid in a template-dependent process, are hybridized to the template strand. Typically, primers are from ten to thirty base pairs in length, but longer sequences can be employed. Primers may be provided in double-stranded and/or single-stranded form, although the single-stranded form generally is preferred.

Often, pairs of primers are designed to selectively hybridize to distinct regions of a template nucleic acid, and are contacted with the template DNA under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

PCR. A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety. In PCR™, pairs of primers that selectively hybridize to nucleic acids are used under conditions that permit selective hybridization. The term primer, as used herein, encompasses any nucleic acid that is capable of priming the synthesis of a nascent nucleic acid in a template-dependent process. Primers may be provided in double-stranded or single-stranded form, although the single-stranded form is preferred.

The primers are used in any one of a number of template dependent processes to amplify the target gene sequences present in a given template sample. One of the best known amplification methods is PCR™ which is described in detail in U.S. Pat. Nos. 4,683,195, 4,683,202 and 4,800,159, each incorporated herein by reference.

In PCR™, two primer sequences are prepared which are complementary to regions on opposite complementary strands of the target-gene(s) sequence. The primers will hybridize to form a nucleic-acid:primer complex if the target-gene(s) sequence is present in a sample. An excess of deoxyribonucleoside triphosphates is added to a reaction mixture along with a DNA polymerase, e.g., Taq polymerase that facilitates template-dependent nucleic acid synthesis.

If the target-gene(s) sequence:primer complex has been formed, the polymerase will cause the primers to be extended along the target-gene(s) sequence by adding on nucleotides. By raising and lowering the temperature of the reaction mixture, the extended primers will dissociate from the target-gene(s) to form reaction products, excess primers will bind to the target-gene(s) and to the reaction products and the process is repeated. These multiple rounds of amplification, referred to as “cycles”, are conducted until a sufficient amount of amplification product is produced.

A reverse transcriptase PCR™ amplification procedure may be performed in order to quantify the amount of mRNA amplified. Methods of reverse transcribing RNA into cDNA are well known and described in Sambrook et al., 2001. Alternative methods for reverse transcription utilize thermostable DNA polymerases. These methods are described in WO 90/07641, filed Dec. 21, 1990.

LCR. Another method for amplification is the ligase chain reaction (“LCR”), disclosed in European Patent Application No. 320,308, incorporated herein by reference. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750, incorporated herein by reference, describes a method similar to LCR for binding probe pairs to a target sequence.

Qbeta Replicase. Qbeta Replicase, described in PCT Patent Application No. PCT/US87/00880, also may be used as still another amplification method in the present invention. In this method, a replicative sequence of RNA, which has a region complementary to that of a target, is added to a sample in the presence of an RNA polymerase. The polymerase will copy the replicative sequence, which can then be detected.

Isothermal Amplification. An isothermal amplification method, in which restriction endonucleases and ligases are used to achieve the amplification of target molecules that contain nucleotide 5′-[α-thio]-triphosphates in one strand of a restriction site also may be useful in the amplification of nucleic acids in the present invention. Such an amplification method is described by Walker et al. 1992, incorporated herein by reference.

Strand Displacement Amplification. Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA.

Cyclic Probe Reaction. Target specific sequences can also be detected using a cyclic probe reaction (CPR). In CPR, a probe having 3′ and 5′ sequences of non-specific DNA and a middle sequence of specific RNA is hybridized to DNA, which is present in a sample. Upon hybridization, the reaction is treated with RNase H, and the products of the probe identified as distinctive products, which are released after digestion. The original template is annealed to another cycling probe and the reaction is repeated.

Transcription-Based Amplification. Other nucleic acid amplification procedures include transcription-based amplification systems (TAS), including nucleic acid sequence based amplification (NASBA) and 3SR, Kwoh et al. (1989); PCT Application WO 88/10315 (each incorporated herein by reference).

In NASBA, the nucleic acids can be prepared for amplification by standard phenol/chloroform extraction, heat denaturation of a clinical sample, treatment with lysis buffer and mini-spin columns for isolation of DNA and RNA or guanidinium chloride extraction of RNA. These amplification techniques involve annealing a primer, which has target specific sequences. Following polymerization, DNA/RNA hybrids are digested with RNase H while double-stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

Other Amplification Methods. Other amplification methods, as described in British Patent Application No. GB 2,202,328, and in PCT Application No. PCT/US89/01025, each incorporated herein by reference, may be used in accordance with the present invention. In the former application, “modified” primers are used in a PCR™ like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Davey et al., European Patent Application No. 329 822 (incorporated herein by reference) disclose a nucleic acid amplification process involving cyclically synthesizing single-stranded RNA (“ssRNA”), ssDNA, and double-stranded DNA (dsDNA), which may be used in accordance with the present invention.

The ssRNA is a first template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA. Miller et al., PCT Patent Application WO 89/06700 (incorporated herein by reference) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts.

Other suitable amplification methods include “race” and “one-sided PCR™” (Frohman, 1990; Ohara et al., 1989, each herein incorporated by reference). Methods based on ligation of two (or more) oligonucleotides in the presence of nucleic acid having the sequence of the resulting “di-oligonucleotide,” thereby amplifying the di-oligonucleotide, also may be used in the amplification step of the present invention, Wu et al., 1989, incorporated herein by reference).

C. Methods for Nucleic Acid Separation

It may be desirable to separate nucleic acid products from other materials, such as template and excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized with light exhibiting the appropriate excitatory spectra.

### V. PERSONAL HISTORY MEASURES AND SCREENING

A. Personal History

In addition to use of the genetic analysis disclosed herein, the present invention may make use of additional factors in gauging an individual's risk for developing cancer. In particular, one will examine multiple factors including age, ethnicity, reproductive history, menstruation history, use of oral contraceptives, body mass index, alcohol consumption history, smoking history, exercise history, and diet to improve the predictive accuracy of the present methods. A history of cancer in a relative, and the age at which the relative was diagnosed with cancer, are also important personal history measures. The inclusion of personal history measures with genetic data in an analysis to predict a phenotype, cancer in this case, is grounded in the realization that almost all phenotypes are derived from a dynamic interaction between an individual's genes and the environment in which these genes act. For example, fair skin may predispose an individual to melanoma but only if the individual is exposed to prolonged unshielded exposure to the sun's ultraviolet radiation. The inventors include personal history measures in their analysis because they are possible modifiers of the penetrance of the cancer phenotype for any genotype examined. Those skilled in the art will realize that the personal history measures listed in this paragraph are unlikely to be the only such environmental factors that affect the penetrance of the cancer phenotype.

B. Self-Examination

All females, regardless of risk factors, should perform monthly self-examinations. It should be performed about the same time each month, optimally the week (2-5 days) after the menstrual period ends, as that is the point when breasts will be the least tender. If an irregular cycle is observed, if there is pregnancy, or post-menopause, the same date each month should be selected.

There are two basic steps to conducting a self-exam: visual observation followed by tactile examination. The visual examination looks for changes in the size, shape, contour, and color of each breast. Also of concern are bumps, lumps, sores and skin changes. Tactile exams should use a pattern to include the breast itself, between the breast and underarm, the underarm itself, and the area above the breast up to the collarbone and across to the shoulder. It is important to check surrounding areas because cancer may be found in the lymph node tissue around your breast and underarm. Using the pads of the three middle fingers, press on the breast with varying degrees of pressure: light (move the skin without moving the tissue underneath), medium (midway into the tissue), and hard (down to the ribs “on the verge of pain”). Patterns include spiral (concentric circles), pie shaped wedges, and up and down. Then repeat the process for the other breast. Once the tactile examination has been performed standing up, it should be done again while lying down.

C. Mammography

Mammography is a specific type of imaging that uses a low-dose x-ray system to examine breasts. A mammography exam, or mammogram, is most often used to aid in the diagnosis of breast diseases in women. Two enhancements to traditional mammography include digital mammography and computer-aided detection. Digital mammography, is a mammography system in which the x-ray film is replaced by solid-state detectors that convert x-rays into electrical signals. The electrical signals are used to produce images of the breast that can be seen on a computer screen or printed on special film similar to conventional mammograms. Computer-aided detection (CAD) systems use a digitized mammographic image that can be obtained from either a conventional film mammogram or a digitally acquired mammogram. The computer software then searches for abnormal areas of density, mass, or calcification that may indicate the presence of cancer.

Mammograms are used as a screening tool to detect early breast cancer in women experiencing no symptoms and to detect and diagnose breast disease in women experiencing symptoms such as a lump, pain or nipple discharge. Mammography is crucial to early detection of breast cancers as it can show changes in the breast up to two years before a patient or physician can feel them. Research has shown that annual mammograms lead to early detection of breast cancers, when they are most curable and breast-conservation therapies are available. The National Cancer Institute (NCI) adds that women who have had breast cancer and those who are at increased risk due to a genetic history of breast cancer should seek expert medical advice about whether they should begin screening before age 40 and about the frequency of screening.

Mammography is performed on an outpatient basis, during which a mammagram-qualified technologist will place the breast on a special platform and compress it with a paddle (often made of clear Plexiglas or other plastic). The compression is necessary in order (i) to even out the breast thickness so that all of the tissue can be visualized, (ii) to spread out the tissue so that small abnormalities won't be obscured by overlying breast tissue, (iii) to allow the use of a lower x-ray dose since a thinner amount of breast tissue is being imaged, (iv) to hold the breast still in order to eliminate blurring of the image caused by motion, and (v) to reduce x-ray scatter to increase sharpness of picture. The examination process takes about 30 minutes.

### VI. KITS

The present invention also contemplates the preparation of kits for use in accordance with the present invention. Suitable kits include various reagents for use in accordance with the present invention in suitable containers and packaging materials, including tubes, vials, and shrink-wrapped and blow-molded packages.

Materials suitable for inclusion in a kit in accordance with the present invention comprises one or more of the following:
gene specific PCR primer pairs (oligonucleotides) that anneal to DNA or cDNA sequence domains that flank the genetic polymorphisms of interest;
reagents capable of amplifying a specific sequence domain in either genomic DNA or cDNA without the requirement of performing PCR;
reagents required to discriminate between the various possible alleles in the sequence domains amplified by PCR or non-PCR amplification (e.g., restriction endonucleases, oligonucleotides that anneal preferentially to one allele of the polymorphism, including those modified to contain enzymes or fluorescent chemical groups that amplify the signal from the oligonucleotide and make discrimination of alleles most robust);
reagents required to physically separate products derived from the various alleles (e.g., agarose or polyacrylamide and a buffer to be used in electrophoresis, HPLC columns, SSCP gels, formamide gels or a matrix support for MALDI-TOF).

### VII. CANCER PROPHYLAXIS

In one aspect of the invention, there is an improved ability to identify candidates for prophylactic cancer treatments due to being identified as at a high genetic risk of developing breast cancer. The primary drugs for use in breast cancer prophylaxis are tamoxifen and raloxifene, discussed further below. However, those skilled in the art will realize that there are other chemopreventative drugs currently under development. The disclosed invention is expected to facilitate more appropriate and effective application of these new drugs also when and if they become commercially available.

A. Tamoxifen

Tamoxifen (NOLVADEX®) a nonsteroidal antiestrogen, is provided as tamoxifen citrate. Tamoxifen citrate tablets are available as 10 mg or 20 mg tablets. Each 10 mg tablet contains 15.2 mg of tamoxifen citrate, which is equivalent to 10 mg of tamoxifen. Inactive ingredients include carboxymethylcellulose calcium, magnesium stearate, mannitol and starch. Tamoxifen citrate is the trans-isomer of a triphenylethylene derivative. The chemical name is (Z)₂-[4-(1,2-diphenyl-1-butenyl)phenoxy]-N,N-dimethylethanamine 2-hydroxy-1,2,3-propanetricarboxylate (1:1). Tamoxifen citrate has a molecular weight of 563.62, the pKa′ is 8.85, the equilibrium solubility in water at 37° C. is 0.5 mg/mL and in 0.02 N HCl at 37° C., it is 0.2 mg/mL.

Tamoxifen citrate has potent antiestrogenic properties in animal test systems. While the precise mechanism of action is unknown, the antiestrogenic effects may be related to its ability to compete with estrogen for binding sites in target tissues such as breast. Tamoxifen inhibits the induction of rat mammary carcinoma induced by dimethylbenzanthracene (DMBA) and causes the regression of DMBA-induced tumors in situ in rats. In this model, tamoxifen appears to exert its anti-tumor effects by binding the estrogen receptors.

Tamoxifen is extensively metabolized after oral administration. Studies in women receiving 20 mg of radiolabeled (¹⁴C) tamoxifen have shown that approximately 65% of the administered dose is excreted from the body over a period of 2 weeks (mostly by fecal route). N-desmethyl tamoxifen is the major metabolite found in patients' plasma. The biological activity of N-desmethyl tamoxifen appears to be similar to that of tamoxifen. 4-hydroxytamoxifen, as well as a side chain primary alcohol derivative of tamoxifen, have been identified as minor metabolites in plasma.

Following a single oral dose of 20 mg, an average peak plasma concentration of 40 ng/mL (range 35 to 45 ng/mL) occurred approximately 5 hours after dosing. The decline in plasma concentrations of tamoxifen is biphasic, with a terminal elimination half-life of about 5 to 7 days. The average peak plasma concentration of N-desmethyl tamoxifen is 15 ng/mL (range 10 to 20 ng/mL). Chronic administration of 10 mg tamoxifen given twice daily for 3 months to patients results in average steady-state plasma concentrations of 120 ng/mL (range 67-183 ng/mL) for tamoxifen and 336 ng/mL (range 148-654 ng/mL) for N-desmethyl tamoxifen. The average steady-state plasma concentrations of tamoxifen and N-desmethyl tamoxifen after administration of 20 mg tamoxifen once daily for 3 months are 122 ng/mL (range 71-183 ng/mL) and 353 ng/mL (range 152-706 ng/mL), respectively. After initiation of therapy, steady state concentrations for tamoxifen are achieved in about 4 weeks and steady state concentrations for N-desmethyl tamoxifen are achieved in about 8 weeks, suggesting a half-life of approximately 14 days for this metabolite.

For patients with breast cancer, the recommended daily dose is 20-40 mg. Dosages greater than 20 mg per day should be given in divided doses (morning and evening). Prophylactic doses may be lower, however.

B. Raloxifene

Raloxifene hydrochloride (EVISTA®) is a selective estrogen receptor modulator (SERM) that belongs to the benzothiophene class of compounds. The chemical designation is methanone, [6-hydroxy-2-(4-hydroxyphenyl)benzo[b]thien-3-yl]-[4-[2-(1-piperidinyl)ethoxy]phenyl]-hydrochloride. Raloxifene hydrochloride (HCl) has the empirical formula C₂₈H₂₇NO₄S.HCl, which corresponds to a molecular weight of 510.05. Raloxifene HCl is an off-white to pale-yellow solid that is very slightly soluble in water.

Raloxifene HCl is supplied in a tablet dosage form for oral administration. Each tablet contains 60 mg of raloxifene HCl, which is the molar equivalent of 55.71 mg of free base. Inactive ingredients include anhydrous lactose, carnuba wax, crospovidone, FD& C Blue No. 2 aluminum lake, hydroxypropyl methylcellulose, lactose monohydrate, magnesium stearate, modified pharmaceutical glaze, polyethylene glycol, polysorbate 80, povidone, propylene glycol, and titanium dioxide.

Raloxifene's biological actions, like those of estrogen, are mediated through binding to estrogen receptors. Preclinical data demonstrate that raloxifene is an estrogen antagonist in uterine and breast tissues. Preliminary clinical data (through 30 months) suggest EVISTA® lacks estrogen-like effects on uterus and breast tissue.

Raloxifene is absorbed rapidly after oral administration. Approximately 60% of an oral dose is absorbed, but presystemic glucuronide conjugation is extensive. Absolute bioavailability of raloxifene is 2.0%. The time to reach average maximum plasma concentration and bioavailability are functions of systemic interconversion and enterohepatic cycling of raloxifene and its glucuronide metabolites.

Following oral administration of single doses ranging from 30 to 150 mg of raloxifene HCl, the apparent volume of distribution is 2.348 L/kg and is not dose dependent. Biotransformation and disposition of raloxifene in humans have been determined following oral administration of ¹⁴C-labeled raloxifene. Raloxifene undergoes extensive first-pass metabolism to the glucuronide conjugates: raloxifene-4′-glucuronide, raloxifene-6-glucuronide, and raloxifene-6, 4′-diglucuronide. No other metabolites have been detected, providing strong evidence that raloxifene is not metabolized by cytochrome P450 pathways. Unconjugated raloxifene comprises less than 1% of the total radiolabeled material in plasma. The terminal log-linear portions of the plasma concentration curves for raloxifene and the glucuronides are generally parallel. This is consistent with interconversion of raloxifene and the glucuronide metabolites.

Following intravenous administration, raloxifene is cleared at a rate approximating hepatic blood flow. Apparent oral clearance is 44.1 L/kg per hour. Raloxifene and its glucuronide conjugates are interconverted by reversible systemic metabolism and enterohepatic cycling, thereby prolonging its plasma elimination half-life to 27.7 hours after oral dosing. Results from single oral doses of raloxifene predict multiple-dose pharmacokinetics. Following chronic dosing, clearance ranges from 40 to 60 L/kg per hour. Increasing doses of raloxifene HCl (ranging from 30 to 150 mg) result in slightly less than a proportional increase in the area under the plasma time concentration curve (AUC). Raloxifene is primarily excreted in feces, and less than 0.2% is excreted unchanged in urine. Less than 6% of the raloxifene dose is eliminated in urine as glucuronide conjugates.

The recommended dosage is one 60 mg tablet daily, which may be administered any time of day without regard to meals. Supplemental calcium is recommended if dietary intake is inadequate.

C. STAR

More than 400 centers across the U.S., Canada and Puerto Rico are currently participating in a clinical trial for tamoxifen and raloxifene, known as STAR. It is one of the largest breast cancer prevention trials ever undertaken. STAR is also the first trial to compare a drug proven to reduce the chance of developing breast cancer with another drug that has the potential to reduce breast cancer risk. All participants receive one or the other drug for five years. At least 22,000 postmenopausal women at high-risk of breast cancer will participate in STAR. All races and ethnic groups are encouraged to participate in STAR.

Tamoxifen (NOLVADEX®) was proven in the Breast Cancer Prevention Trial to reduce breast cancer incidence by 49 percent in women at increased risk of the disease. The U.S. Food and Drug Administration (FDA) approved the use of tamoxifen to reduce the incidence of breast cancer in women at increased risk of the disease in October 1998. Tamoxifen has been approved by the FDA to treat women with breast cancer for more than 20 years and has been in clinical trials for about 30 years.

Raloxifene (trade name EVISTA®) was shown to reduce the incidence of breast cancer in a large study of its use to prevent and treat osteoporosis. This drug was approved by the FDA to prevent osteoporosis in postmenopausal women in December 1997 and has been under study for about five years.

The study is a randomized double-blinded clinical trial to compare the effectiveness of raloxifene with that of tamoxifen in preventing breast cancer in postmenopausal women. Women must be at least 35 years old, have gone no more than one year since undergoing mammography with no evidence of cancer, have no previous mastectomy to prevent breast cancer, have no previous invasive breast cancer or intraductal carcinoma in situ, have not had hormone therapy in at least three months, and have no previous radiation therapy to the breast.

Patients will be randomly assigned to one of two groups. Patients in group one will receive raloxifene plus a placebo by mouth once a day. Patients in group two will receive tamoxifen plus a placebo by mouth once a day. Treatment will continue for 5 years. Quality of life will be assessed at the beginning of the study and then every 6 months for 5 years. Patients will then receive follow-up evaluations once a year.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Methods

Mathematical Model. The inventor developed a mathematical model for the estrogen metabolism pathway shown in FIG. 1. He assume that each reaction in the pathway (A→B, a generic step in the pathway) is an enzyme-catalyzed reaction of the form:

$$A + E \begin{matrix}{}^{{k}_{1}} \\ {}_{{k}_{2}}\end{matrix} C {}^{{k}_{3}} B + E ,$$

where E denotes the enzyme, C is the enzyme-substrate complex, and kᵢ, i=1,2,3, are the rate constants of the reaction. For these types of reaction, the inventor approximated the kinetics using the quasi-steady state assumption:

$$C = \frac{{E}^{*} A}{{K}_{m} + A} , {K}_{m} = \frac{{k}_{2} + {k}_{3}}{{k}_{1}} ,$$

where E* is the initial enzyme concentration. With this assumption, one has:

$$\frac{d B}{d t} \approx \frac{{k}_{cat} {E}^{*} A}{{K}_{m} + A}$$

where k_{cat} is a constant. This approach leads to a system of nonlinear, ordinary differential equations for the concentrations of the compounds in the pathway. In each equation, k_{cat}_{ⱼ} and Kₘ_{ⱼ} are constants and E_{enzyme} are the enzyme levels in the respective reactions.

$$\begin{matrix}\frac{d ( {E}_{2} )}{d t} = - \frac{{k}_{{cat}_{1}} {E}_{CYP 1 B 1} {E}_{2}}{{K}_{{m}_{1}} + {E}_{2}} - \frac{{k}_{{cat}_{2}} {E}_{CYP 1 A 1} {E}_{2}}{{K}_{{m}_{2}} + {E}_{2}} - \frac{{k}_{{cat}_{3}} {E}_{CYP 1 B 1} {E}_{2}}{{K}_{{m}_{3}} + {E}_{2}} & ( 1 ) \\ \frac{d ( {OHE}_{2}^{2} )}{d t} = \frac{{k}_{{cat}_{2}} {E}_{CYP 1 A 1} {E}_{2}}{{K}_{{m}_{2}} + {E}_{2}} + \frac{{k}_{{cat}_{3}} {E}_{CYP 1 B 1} {E}_{2}}{{K}_{{m}_{3}} + {E}_{2}} - \frac{{k}_{{cat}_{6}} {E}_{COMT} {OHE}_{2}^{2}}{{K}_{{m}_{6}} + {OHE}_{2}^{2}} + \frac{{k}_{{cat}_{7}} {E}_{CYP 1 A 1} {MeOHE}_{2}^{2}}{{K}_{{m}_{7}} + {MeOHE}_{2}^{2}} + \frac{{k}_{{cat}_{8}} {E}_{CYP 1 B 1} {MeOHE}_{2}^{2}}{{K}_{{m}_{8}} + {MeOHE}_{2}^{2}} - \frac{{k}_{cat 9} {E}_{COMT} {OHE}_{2}^{2}}{{K}_{{m}_{9}} + {OHE}_{2}^{2}} + \frac{{k}_{{cat}_{10}} {E}_{CYP 1 A 1} {MeOHE}_{2}^{23}}{{K}_{{m}_{10}} + {MeOHE}_{2}^{23}} + \frac{{k}_{{cat}_{11}} {E}_{CYP 1 B 1} {MeOHE}_{2}^{23}}{{K}_{{m}_{11}} + {MeOHE}_{2}^{23}} - \frac{{{V}_{{max}_{Q 1}} ( {OHE}_{2}^{2} )}^{{σ}_{{Q}_{1}}}}{{K}_{{m}_{Q 1}} + {( {OHE}_{2}^{2} )}^{{σ}_{{Q}_{1}}}} & ( 2 ) \\ \frac{d ( {OHE}_{2}^{4} )}{d t} = \frac{{k}_{{cat}_{1}} {E}_{CYP 1 B 1} {E}_{2}}{{K}_{{m}_{1}} + {E}_{2}} - \frac{{k}_{{cat}_{4}} {E}_{COMT} {OHE}_{2}^{4}}{{K}_{{m}_{4}} + {OHE}_{2}^{4}} + \frac{{k}_{{cat}_{5}} {E}_{COMT} {MeOHE}_{2}^{4}}{{K}_{{m}_{5}} + {MeOHE}_{2}^{4}} - \frac{{{V}_{{max}_{Q 2}} ( {OHE}_{2}^{4} )}^{{σ}_{{Q}_{2}}}}{{K}_{{m}_{Q 2}} + {( {OHE}_{2}^{4} )}^{{σ}_{{Q}_{2}}}} & ( 3 ) \\ \frac{d ( {MeOHE}_{2}^{4} )}{d t} = \frac{{k}_{{cat}_{4}} {E}_{COMT} {OHE}_{2}^{4}}{{K}_{{m}_{4}} + {OHE}_{2}^{4}} - \frac{{k}_{{cat}_{5}} {E}_{CYP 1 B 1} {MeOHE}_{2}^{4}}{{K}_{{m}_{5}} + {MeOHE}_{2}^{4}} & ( 4 ) \\ \frac{d ( {MeOHE}_{2}^{2} )}{d t} = \frac{{k}_{{cat}_{6}} {E}_{COMT} {OHE}_{2}^{2}}{{K}_{{m}_{6}} + {OHE}_{2}^{2}} - \frac{{k}_{{cat}_{7}} {E}_{CYP 1 A 1} {MeOHE}_{2}^{2}}{{K}_{{m}_{7}} + {MeOHE}_{2}^{2}} - \frac{{k}_{{cat}_{8}} {E}_{CYP 1 B 1} {MeOHE}_{2}^{2}}{{K}_{{m}_{8}} + {MeOHE}_{2}^{2}} & ( 5 ) \\ \frac{d ( {MeOHE}_{2}^{23} )}{d t} = \frac{{k}_{cat 9} {E}_{COMT} {OHE}_{2}^{2}}{{K}_{{m}_{9}} + {OHE}_{2}^{2}} - \frac{{k}_{{cat}_{10}} {E}_{CYP 1 A 1} {MeOHE}_{2}^{23}}{{K}_{{m}_{10}} + {MeOHE}_{2}^{23}} - \frac{{k}_{{cat}_{11}} {E}_{CYP 1 B 1} {MeOHE}_{2}^{23}}{{K}_{{m}_{11}} + {MeOHE}_{2}^{23}} & ( 6 ) \\ \frac{d ( {EQ}_{2}^{23} )}{d t} = \frac{{{V}_{{max}_{Q 1}} ( {OHE}_{2}^{2} )}^{{σ}_{{Q}_{1}}}}{{K}_{{m}_{Q 1}} + {( {OHE}_{2}^{2} )}^{{σ}_{{Q}_{1}}}} - \frac{{k}_{{cat}_{13}} {E}_{GSTP 1} {EQ}_{2}^{23}}{{K}_{{m}_{13}} + {EQ}_{2}^{23}} - \frac{{k}_{{cat}_{14}} {E}_{GSTP 1} {EQ}_{2}^{23}}{{K}_{{m}_{14}} + {EQ}_{2}^{23}} - {k}_{1} {EQ}_{2}^{23} & ( 7 ) \\ \frac{d ( {EQ}_{2}^{34} )}{d t} = \frac{{{V}_{{max}_{Q 2}} ( {OHE}_{2}^{4} )}^{{σ}_{{Q}_{2}}}}{{K}_{{m}_{Q 2}} + {( {OHE}_{2}^{4} )}^{{σ}_{{Q}_{2}}}} - \frac{{k}_{{cat}_{12}} {E}_{GSTP 1} {EQ}_{2}^{34}}{{K}_{{m}_{12}} + {EQ}_{2}^{34}} - {k}_{1} {EQ}_{2}^{34} & ( 8 ) \\ \frac{d ( {OHE}_{2}^{21} SG )}{d t} = \frac{{k}_{{cat}_{14}} {E}_{GSTP 1} {EQ}_{2}^{23}}{{K}_{{m}_{14}} + {EQ}_{2}^{23}} & ( 9 ) \\ \frac{d ( {OHE}_{2}^{24} SG )}{d t} = \frac{{k}_{{cat}_{13}} {E}_{GSTP 1} {EQ}_{2}^{23}}{{K}_{{m}_{13}} + {EQ}_{2}^{23}} & ( 10 ) \\ \frac{d ( {OHE}_{2}^{42} SG )}{d t} = \frac{{k}_{{cat}_{12}} {E}_{GSTP 1} {EQ}_{2}^{34}}{{K}_{{m}_{12}} + {EQ}_{2}^{34}} & ( 11 )\end{matrix}$$

There are parts of the pathway for which kinetic data are not available. In particular, rate constants cannot be determined experimentally for the reaction sequences 2-OHE₂→E₂-2,3-SQ→E₂→2,3-Q and 4-OHE₂→E₂-3,4-SQ→E₂-3,4-Q because of the transient nature of the semiquinones (msec half-life) (Kalyanaraman et al., 1984). Therefore, the inventor simplified the pathway and collapsed the sequential reactions to single reactions, 2-OHE₂→E₂-2,3-Q and 4-OHE₂→E₂-3,4-Q, respectively. The inventor also assumed that each of these quinone production reactions (OHE₂^{k}→EQ₂^{ij}) satisfies dynamics of the form:

$$\frac{d {EQ}_{2}^{ij}}{d t} = \frac{{{V}_{{max}_{Q}} ( {OHE}_{2}^{k} )}^{{σ}_{Q}}}{{K}_{{m}_{Q}} + {( {OHE}_{2}^{k} )}^{{σ}_{Q}}}$$

where Vₘₐₓ_{_{Q}}, Kₘ_{_{Q}} and σ_{Q} are constants. For the mathematical model to be a tractable computational model of the metabolism pathway, it is necessary to have estimates of these unknown constants. The inventor used two types of experimental data to derive the constants Vₘₐₓ_{_{Q1}}, Vₘₐₓ_{_{Q2}}, Kₘ_{_{Q1}}, Kₘ_{_{Q2}}, σ_{Q}_{₁} and σ_{Q}_{₂}. First, he used rate constants determined experimentally for individual reactions catalyzed by CYP1A1, CYP1B1, COMT, and GSTP1 (Hanna et al., 2000; Dawling et al., 2001; Hachey et al., 2003; Dawling et al., 2003). Second, he used the concentrations over time determined for every non-quinone compound in the pathway following simultaneous incubation of the parent hormone E₂ with all four enzymes (Dawling et al., 2004). Using the experimental data, a searching algorithm was written in Mathematica (Wolfram Research, Inc., Champaign, Ill.) to find values for Vₘₐₓ_{_{Q}}, Kₘ_{_{Q}} and σ_{Q}. The derived parameters, Vₘₐₓ_{_{Q1}}, Vₘₐₓ_{_{Q2}}, Kₘ_{_{Q1}}, Kₘ_{_{Q2}}, σ_{Q}_{₁}, and σ_{Q}_{₂}, for the two quinone reactions were chosen to fit the experimental data using numerical solutions of the system of differential equation.

As a measure of the quinone concentrations over the course of time, the inventor introduced the area under the curve (AUC) metric:

$$A U {C}_{k} = {\int }_{0}^{T} {EQ}_{2}^{k} ( t ) d t ,$$

where k=23,34 and T=30 min. It is possible to introduce other measures, e.g.,

$${EQ}_{2 max}^{ij} = {max}_{0 \leq t \leq T} {EQ}_{2}^{ij} ( t ) ,$$

which is the highest concentration achieved during the time interval [0,T]. The chose the former metric because it incorporates both concentration and time.

CYP1A1 Variants. Wild-type CYP1A1 cDNA was prepared for expression and purification of recombinant CYP1A1 as described previously (Hanna et al., 2000; Dawling et al., 2003). Site-directed mutagenesis was performed to generate the cDNA variants, which were verified by nucleotide sequence analysis and then similarly expressed and purified: 462Ile→Val (m2), 461Thr→Asn (m4), and 461Asn-462Val (m2/m4) (Cascorbi et al., 1996). SDS-polyacrylamide gel electrophoresis showed >95% protein purity and the reduced-CO difference spectrum revealed the λₘₐₓ at 450 nm, which allowed quantitation for subsequent enzyme experiments. The inventor used GC/MS (Hanna et al., 2000; Dawling et al., 2003) to determine the reaction kinetics of E₂ oxidation for wild-type, m2, m4, and m2/m4 CYP1A1.

Study Population. The hospital-based case-control study group of 221 Caucasian women with primary invasive breast cancer and their age-matched control subjects has been described previously (Ritchie et al., 2001; Bailey et al., 1998; Bailey et al., 1998). Genomic DNA was extracted from tumor tissue or WBCs. The DNA samples of four control subjects had been depleted, leaving 221 cases and 217 controls for the study group.

DNA Analysis. The genotypes of CYP1A1, CYP1B1, and COMT were determined by PCR and restriction endonuclease digestion as previously described (Dawling et al., 2001; Bailey et al., 1998; Bailey et al., 1998). Each PCR contained internal controls for the respective gene and random retesting of 50 samples yielded 100% reproducibility. Direct sequencing of 5 different samples provided further independent genotype validation.

Statistical Analysis. The Wilcoxon rank sum test was used to determine the median difference in age between cases and controls. The Ω² test was used to compare the distribution of CYP1A1, CYP1B1, and COMT alleles in cases and controls. The Ω² goodness-of-fit test was used for testing Hardy-Weinberg equilibrium. Haplotype frequencies were estimated via the expectation-maximization algorithm in Powermarker 3.23 (Liu and muse, www.powermarker.net; Excoffier and Slatkin, 1995). Haplotype-trait association with breast cancer was tested using a regression approach also in Powermarker 3.23 (Liu and muse, www.powermarker.net; Zaykin et al., 2002).

Composite E₂-3,4-Q AUC. There are 4 CYP1A1, 16 CYP1B1, and 2 COMT (Dawling et al., 2001; Bailey et al., 1998; Bailey et al., 1998) haplotypes with 4×16×2=128 possible genetic combinations. The inventor calculated an E₂-3,4-Q AUC for each woman based on her CYP1A1, CYP1B1, and COMT haplotypes, which together the inventor terms a composite haplotype. In calculating each AUC, he considered that the CYP1B1 gene has four polymorphic sites with 16 possible haplotypes. Since any individual can possess only two haplotypes, the certainty of assigning the correct CYP1B1 haplotypes becomes a matter of probability if the individual is heterozygous for more than one polymorphic site. The probability of a particular composite haplotype occurring in our population of 438 women was computed in the following manner. For each individual, a subset of the 128 possible combinations was computed resulting in 438 subsets of haplotype combinations (CYP1A1, CYP1B1 and COMT). Then a count was performed for each of the 128 possible composite haplotypes in the 438 subsets from which a frequency chart was constructed. From this chart, the inventor defined the probabilities of composite haplotypes occurring in the population. Suppose an individual has n possible composite haplotypes of CYP1A1, CYP1B, and COMT and let AUC₁, AUC₂, . . . , AUCₙ denote the area under the curve values for each haplotype. If the probabilities that these composite haplotypes occur in the population are, P₁, P₂, . . . , Pₙ where 0<Pᵢ<1, i=1, 2, . . . , n, then the composite AUC_{comp} is defined as

$$A U {C}_{comp} = \frac{{\sum }_{i = 1}^{n} {P}_{i} A U {C}_{i}}{{\sum }_{i = 1}^{n} {P}_{i}} .$$

### Example 2

### Results

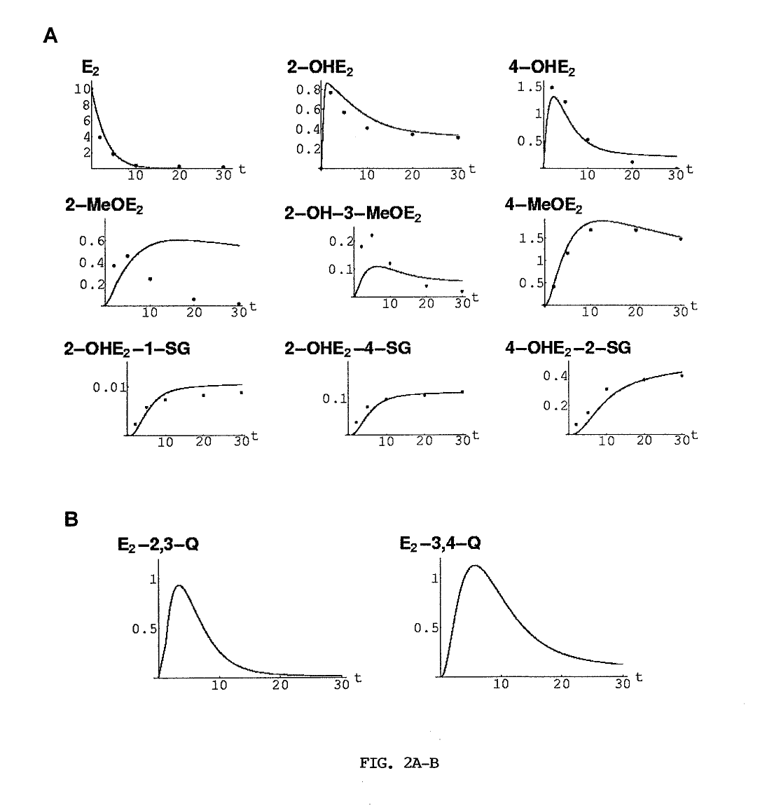

Validation of in silico Model against Experimental Data. In a previous study, the inventor determined the metabolism of E₂, 2-OHE₂, 4-OHE₂, 2-MeOE₂, 2-OH-3-MeOE₂, 4-MeOE₂, 2-OHE₂-1-SG, 2-OHE₂-4-SG, and 4-OHE₂-2-SG as a function of time in the presence of CYP1A1 (85 pmol), CYP1B1 (165 pmol), COMT (125 pmol), and GSTP1 (500 pmol). Each experimental reaction contained 10 μM E₂, 100 μM S-adenosyl methionine, 100 μM glutathione, and proceeded for 0, 2, 5, 10, 20, and 30 min at 37° C., followed by GC/MS and LC/MS analysis (Dawling et al., 2004). FIG. 2A shows superimposed the experimental data (dots) and the model simulations (curves) for all nine analytes over the 30 min reaction time. In the simulations it was assumed that initially all analyte concentrations are zero, except E₂(0)=E₂*. Enzyme concentrations used in the simulations are consistent with those used in the preceding experimental studies (Hanna et al., 2000; Dawling et al., 2001; Hachey et al., 2003; Dawling et al., 2004). Given the complexity of the pathway, there is excellent agreement between the simulated and experimental results. Of the nine analytes, only two, 2-MeOE₂ and 2-OH-3-MeOE₂, showed a noticeable difference between simulated and measured results. As shown in FIG. 1, the likely reason for this discrepancy lies in the more complex kinetics of 2-MeOE₂ and 2-OH-3-MeOE₂. These methoxyestrogens are the only analytes that are subject to the simultaneous action of three enzymes, i.e., the COMT-mediated production, which is counteracted by CYP1A1- and CYP1B1-mediated demethylation (Dawling et al., 2003.

Estrogen Quinones. The estrogen quinones are too labile to be reliably quantified in a multi-enzyme system. However, as outlined in ‘Methods’, the inventor could use the mathematical model to provide functional relations between E₂(t) and the estrogen quinone concentrations: EQ₂²³(t) and EQ₂³⁴(t). FIG. 2B shows the simulated production and disappearance of the estrogen quinones during the 30 min reaction with a lower level and faster, nearly complete disappearance of E₂-2,3-Q compared to the higher, more sustained level of E₂-3,4-Q.

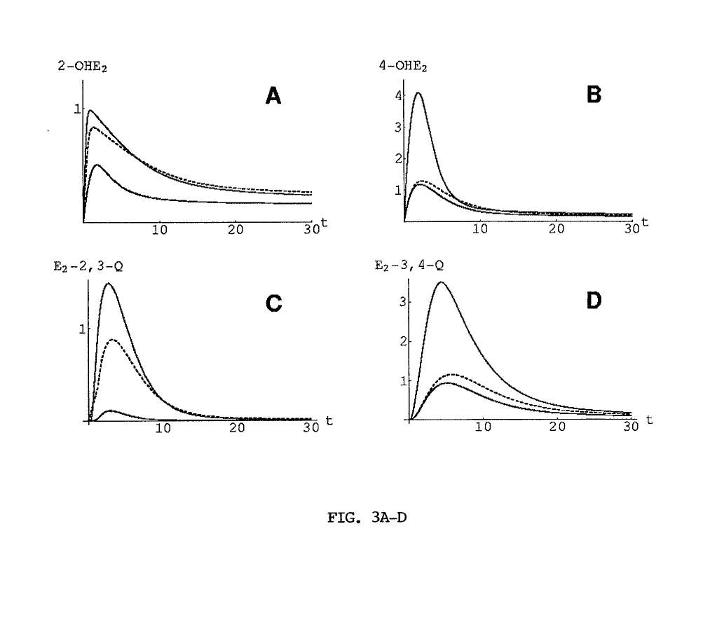

Enzyme Polymorphisms. Since the model was built on experimentally determined rate constants, the inventor could analyze how variations in these kinetic parameters, occurring as the result of enzyme polymorphisms, affect single steps or a combination of steps in the pathway. Table 1 summarizes the kinetic parameters determined for variants of CYP1A1 (this study), CYP1B1 (Lewis et al., 2003 and COMT (Dawling et al., 2001). The inventor applied the model to simulate reactions using the rate constants for 4 CYP1A1, 16 CYP1B1, and 2 COMT wild-type and variant enzymes. For each of the 4×16×2=128 possible genetic combinations, the inventor simulated values for the resulting estrogen metabolites over the 30 min reaction time and then used interpolatory polynomials for these functions to calculate the respective AUCs. These simulations permitted the inventor to see that the combinations of enzyme variants produced a continuous spectrum of concentrations over time for each of the estrogen metabolites. Accordingly, the model allowed the inventor to identify which variant combinations of CYP1A1, CYP1B1, and COMT produced the highest or lowest estrogen metabolite concentrations over time. Since the catechols and quinones have been shown to cause DNA damage, the inventor focused his attention on these two groups of metabolites (FIGS. 3A-D). Of the 128 combinations of CYP1A1, CYP1B1, and COMT variants, he found, for example, that the haplotype combination CYP1A1_{461Asn-462Ile}CYP1B1_{48Arg-119Ser-432Val-453Asn}COMT_{108Met} produced the maximum AUC for both 4-OHE₂ and E₂-3,4-Q (FIGS. 3B,D).

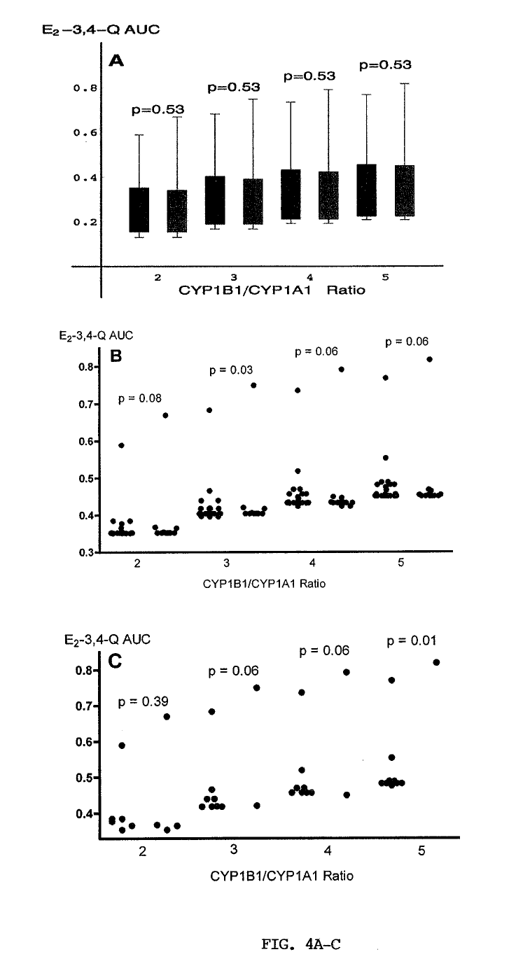

Clinical Application of Kinetic-Genomic Model. The applied the model to a hospital-based breast cancer case-control population that has been analyzed previously (Ritchie et al., 2001; Bailey et al., 1998; Bailey et al., 1998). The two principal differences to the preceding studies are (i) the evaluation of haplotypes rather than genotypes and (ii) the integrated examination of CYP1A1, CYP1B1, and COMT instead of as independent entities. Table 2 summarizes the allele and haplotype data of the case-control population. Only three of the four possible CYP1A1 haplotypes and 12 of 16 possible CYP1B1 haplotypes were observed in the study group. Among the 12 CYP1B1 haplotypes, 8 were present in both cases and controls. Three of the uncommon haplotypes were seen only in cases and one rare haplotype was found only in controls. The overall p-value for the CYP1B1 haplotype distribution among cases and controls was 0.63. In the simulations, the inventor focused the analysis on the E₂-3,4-Q AUC because E₂-3,4-Q has been identified as the principal estrogen metabolite causing DNA damage (Li et al., 2004; Liehr et al., 1986; Li and Li, 1987; Embrechts et al., 2003). The inventor calculated a composite E₂-3,4-Q AUC for each woman based on her CYP1A1, CYP1B1, and COMT haplotypes as outlined above. This information was then used to rank every woman in the entire study population based on her individual E₂-3,4-Q AUC (FIGS. 4A-C).

A major weakness of genetic studies is the neglect of phenotypic factors. This is particularly true for polymorphic enzymes whose activity levels can vary considerably more in response to inducing agents than as a result of a single inherited amino acid substitution. For this reason, the inventor considered the effect of changing the concentration of the phase I enzymes, which play the principal role in the metabolic pathway. As shown in FIGS. 4A-C, the inventor varied the CYP1B1/CYP1A1 ratio from 2 to 5 in the model to reflect reported 4-OHE₂/2-OHE₂ ratios in breast tissue (Liehr and Ricci, 1996; Castagnetta et al., 2002; Rogan et al., 2003). In these simulations, the inventor changed the concentrations of CYP1B1 while keeping CYP1A1 constant. The concentrations of COMT and GSTP1 remained unchanged. When the inventor ranked the E₂-3,4-Q AUCs for the entire study population at different CYP1B1/CYP1A1 ratios, the inventor observed a rise in median AUCs for cases and controls with increasing CYP ratio (FIG. 4A). There were no significant differences between case and control E₂-3,4-Q AUCs at any CYP1B1/CYP1A1 ratio. However, cases predominated in the top tier of the population as shown for the top 8 percentile (35 subjects) of the study group (FIG. 4B). At CYP1B1/CYP1A1=5 the model identified 23 cases and 12 controls (p=0.06). The discriminating ability was even more pronounced in the top 2 percentile (10 subjects) of the study population (FIG. 4C). At CYP1B1/CYP1A1=5 there were 9 cases and 1 control (p=0.01). Table 3 summarizes the composite CYP1A1, CYP1B1, and COMT haplotypes together with the E₂-3,4-Q AUCs for the top 10 women.

TABLE 1
Kinetic parameters for wt and variant CYP1A1, CYP1B1 &COMT

| Reaction | Enzyme | Allele | k_{cat} or Vₘₐₓ | Kₘ |
|---|---|---|---|---|
| E₂ → 2-OHE₂ | CYP1A1 | 461Thr-462Ile (wt*) | 1.50 | 17 |
| | | 461Asn-462Ile | 1.10 | 23 |
| | | 461Thr-462Val | 3.60 | 18 |
| | | 461Asn-462Val | 1.70 | 23 |
| E₂ → 2-OHE₂ | CYP1B1 | 48Arg-119Ala-432Val-453Asn (wt) | 0.36 | 24 |
| | | 48Gly-119Ala-432Val-453Asn | 0.17 | 12 |
| | | 48Arg-119Ser-432Val-453Asn | 0.29 | 19 |
| | | 48Arg-119Ala-432Leu-453Asn | 0.35 | 17 |
| | | 48Arg-119Ala-432Val-453Ser | 0.91 | 49 |
| | | 48Gly-119Ser-432Leu-453Ser | 0.50 | 33 |
| | | 48Arg-119Ala-432Leu-453Ser | 0.56 | 30 |
| | | 48Gly-119Ser-432Leu-453Asn | 0.28 | 17 |
| | | 48Gly-119Ser-432Val-453Asn | 0.19 | 13 |
| | | 48Gly-119Ser-432Val-453Ser | 0.64 | 34 |
| | | 48Arg-119Ser-432Leu-453Asn | 0.56 | 33 |
| | | 48Arg-119Ser-432Leu-453Ser | 0.47 | 24 |
| | | 48Gly-119Ala-432Leu-453Ser | 0.47 | 31 |
| | | 48Gly-119Ala-432Leu-453Asn | 0.19 | 9.1 |
| | | 48Arg-119Ser-432Val-453Ser | 0.65 | 36 |
| | | 48Gly-119Ala-432Val-453Ser | 0.23 | 75 |
| E₂ → 4-OHE₂ | CYP1B1 | 48Arg-119Ala-432Val-453Asn (wt) | 2.10 | 14 |
| | | 48Gly-119Ala-432Val-453Asn | 0.80 | 6.6 |
| | | 48Arg-119Ser-432Val-453Asn | 1.70 | 9.3 |
| | | 48Arg-119Ala-432Leu-453Asn | 1.40 | 9.6 |
| | | 48Arg-119Ala-432Val-453Ser | 3.10 | 21 |
| | | 48Gly-119Ser-432Leu-453Ser | 1.70 | 15 |
| | | 48Arg-119Ala-432Leu-453Ser | 2.20 | 13 |
| | | 48Gly-119Ser-432Leu-453Asn | 0.71 | 5.8 |
| | | 48Gly-119Ser-432Val-453Asn | 1.10 | 5.5 |
| | | 48Gly-119Ser-432Val-453Ser | 2.20 | 15 |
| | | 48Arg-119Ser-432Leu-453Asn | 1.90 | 15 |
| | | 48Arg-119Ser-432Leu-453Ser | 1.90 | 13 |
| | | 48Gly-119Ala-432Leu-453Ser | 1.80 | 12 |
| | | 48Gly-119Ala-432Leu-453Asn | 0.73 | 7.2 |
| | | 48Arg-119Ser-432Val-453Ser | 2.70 | 17 |
| | | 48Gly-119Ala-432Val-453Ser | 0.81 | 28 |
| 2-OHE₂ → 2-MeOE₂ | COMT | 108Val (wt) | 6.80 | 117 |
| | | 108Met | 2.72 | 99 |
| 2-OHE₂ → 2-OH-3-MeOE₂ | COMT | 108Val (wt) | 1.50 | 51 |
| | | 108Met | 0.62 | 58 |
| 4-OHE₂ → 4-MeOE₂ | COMT | 108Val (wt) | 3.40 | 24 |
| | | 108Met | 1.94 | 28 |

*wt = wild-type

TABLE 2
CYP1A1, CYP1B1 & COMT allele and haplotype
freq. of age-matched study pop.

| | Cases | Controls | p-value |
|---|---|---|---|
| Number | 221 | 217 | |

Age (years)

| | | | |
|---|---|---|---|
| Mean | 57.4 | 57.3 | 0.99 |
| Median | 56 | 57 | |

Allele Frequency
CYP1A1

| | | | | |
|---|---|---|---|---|
| Codon 461 | Thr | 0.955 | 0.956 | |
| | Asn | 0.045 | 0.044 | 0.916 |
| | HWE* | 1.000 | 1.000 | |
| Codon 462 | Ile | 0.950 | 0.963 | |
| | Val | 0.050 | 0.037 | 0.348 |
| | HWE | 0.011 | 0.245 | |

CYP1B1

| | | | | |
|---|---|---|---|---|
| Codon 48 | Arg | 0.661 | 0.684 | |
| | Gly | 0.339 | 0.316 | 0.455 |
| | HWE | 1.000 | 0.871 | |
| Codon 119 | Ala | 0.649 | 0.682 | |
| | Ser | 0.351 | 0.318 | 0.305 |
| | HWE | 0.463 | 0.633 | |
| Codon 432 | Val | 0.423 | 0.433 | |
| | Leu | 0.577 | 0.567 | 0.763 |
| | HWE | 0.205 | 0.078 | |
| Codon 453 | Asn | 0.824 | 0.823 | |
| | Ser | 0.176 | 0.177 | 0.971 |
| | HWE | 0.362 | 0.657 | |

COMT

| | | | | |
|---|---|---|---|---|
| Codon 108 | Val | 0.516 | 0.505 | |
| | Met | 0.484 | 0.495 | 0.740 |
| | HWE | 0.358 | 0.889 | |

Haplotype frequency

| | | | |
|---|---|---|---|
| CYP1A1 | | | 0.411 |
| 461Thr-462Ile | 0.905 | 0.919 | |
| 461Thr-462Val | 0.045 | 0.037 | |
| 461Asn-462Ile | 0.050 | 0.044 | |
| CYP1B1 | | | 0.630 |
| 48Arg-119Ala-432Val-453Asn | 0.379 | 0.380 | |
| 48Gly-119Ser-432Leu-453Asn | 0.302 | 0.261 | |
| 48Arg-119Ala-432Leu-453Ser | 0.164 | 0.151 | |
| 48Arg-119Ala-432Leu-453Asn | 0.104 | 0.139 | |
| 48Gly-119Ser-432Val-453Asn | 0.029 | 0.040 | |
| 48Gly-119Ser-432Leu-453Ser | 0.004 | 0.011 | |
| 48Arg-119Ala-432Val-453Ser | 0.003 | 0.011 | |
| 48Arg-119Ser-432Leu-453Ser | 0.001 | 0.005 | |
| 48Arg-119Ser-432Val-453Asn | 0.007 | n.o.** | |
| 48Gly-119Ser-432Val-453Ser | 0.004 | n.o. | |
| 48Arg-119Ser-432Leu-453Asn | 0.003 | n.o. | |
| 48Glv-119Ala-432Val-453Asn | n.o. | 0.002 | |

*HWE, Hardy-Weinberg equilibrium;
**n.o. = not observed

TABLE 3
Composite CYP1A1, CYP1B1 & COMT haplotypes of women with top
10 E₂-3,4-Q AUC values at CYP1B1/CYP1A1 ratio = 5

| Rank | AUC | Subject | Age | CYP1A1 | CYP1B1 | COMT |
|---|---|---|---|---|---|---|
| 1 | 0.8069 | control | 81 | Asn-IIe | Arg-Ala-Val-Asn | Met |
| | | | | Asn-IIe | Arg-Ala-Leu-Asn | Met |
| 2 | 0.7542 | case | 86 | Asn-IIe | Arg-Ala-Leu-Asn | Val |
| | | | | Asn-IIe | Arg-Ala-Leu-Ser | Val |
| | | | | Asn-IIe | Arg-Ala-Leu-Asn | Met |
| | | | | Asn-IIe | Arg-Ala-Leu-Ser | Met |
| 3 | 0.5381 | case | 68 | Asn-IIe | Arg-Ala-Leu-Asn | Val |
| | | | | Asn-IIe | Gly-Ser-Leu-Asn | Val |
| | | | | Asn-IIe | Arg-Ser-Leu-Asn | Val |
| | | | | Asn-IIe | Gly-Ala-Leu-Asn | Val |
| 4 | 0.4803 | case | 76 | Thr-Ile | Arg-Ala-Leu-Asn | Met |
| | | | | Thr-Val | Arg-Ala-Leu-Asn | Met |
| 5 | 0.4800 | case | 47 | Thr-Ile | Arg-Ala-Val-Asn | Met |
| | | | | Asn-Ile | Arg-Ala-Val-Asn | Met |
| 6 | 0.4709 | case | 59 | Thr-Ile | Arg-Ala-Leu-Ser | Met |
| | | | | Thr-Ile | Arg-Ala-Leu-Ser | Val |
| 7 | 0.4709 | case | 45 | Thr-Ile | Arg-Ala-Leu-Ser | Met |
| | | | | Thr-Ile | Arg-Ala-Leu-Ser | Val |
| 8 | 0.4709 | case | 53 | Thr-Ile | Arg-Ala-Leu-Ser | Met |
| | | | | Thr-Ile | Arg-Ala-Leu-Ser | Val |
| 9 | 0.4709 | case | 48 | Thr-Ile | Arg-Ala-Leu-Ser | Met |
| | | | | Thr-Ile | Arg-Ala-Leu-Ser | Val |
| 10 | 0.4617 | case | 56 | Thr-Ile | Arg-Ala-Leu-Ser | Val |
| | | | | Asn-IIe | Arg-Ala-Leu-Ser | Val |

### Example 3

### Discussion

The complexity of mammary estrogen metabolism was recognized several years ago and outlined in a qualitative model (Newbold and Liehr, 2000; Yager and Liehr, 1996). While this model defined the role of specific components, e.g., the oxidizing phase I and conjugating phase II enzymes, the quantitative impact of these enzymes in the overall pathway could not be assessed. The experimental analysis of single enzymes with simple substrate-product kinetics offered an incomplete picture of the pathway limited to the enzyme examined. Here, the inventor presents a new approach that incorporates experimental data previously obtained with individual enzymes into a mathematical model of the estrogen metabolism pathway. Instead of simply performing a parametric fitting exercise, actual experimental rate constants were used to develop the model, which consists of eleven differential equations that permit us to simulate the kinetics of E₂ and eight metabolites in the multi-enzyme pathway. The model simulations were validated against experimental results obtained previously by incubating E₂ with the combined enzymes CYP1A1, CYP1B1, COMT, and GSTP1 (Dawling et al., 2004) and showed excellent concordance of simulated and measured results. Of the nine analytes, only 2-MeOE₂ and 2-OH-3-MeOE₂, showed a noticeable deviation of the simulated from the measured results, most likely due to their more complex kinetics resulting from the simultaneous involvement of three enzymes, COMT, CYP1A1, CYP1B1. It is noteworthy that the deviation of the two methoxyestrogens did not affect the simulation of more distal metabolites, such as the GSH-estrogen conjugates, which showed excellent agreement (FIG. 2A).

Catechol estrogens and estrogen quinones occupy pivotal positions in the oxidative estrogen metabolism pathway (FIG. 1). Using GC/MS, one can follow the production and disappearance of the catechol estrogens (FIG. 2A). Ideally, measurements of the estrogen quinones should be made, but they are highly reactive with short half-lives (seconds to minutes) due to the strained 1,2-diketone functionality inherent in o-quinones (Tabakovic et al., 1996). While estrogen quinones are too labile to be reliably quantified in a multi-enzyme system, the model allowed us to simulate their production and disappearance during the 30 min reaction (FIG. 2B). The disappearance of the quinones is due to two factors, the conversion of the quinones into stable GSH-estrogen conjugates and the irreversible loss from the system, most likely due to binding of the reactive quinones to protein (Tabakovic and Abul-Hajj, 1994). The more rapid disappearance of E₂-2,3-Q compared to E₂-3,4-Q is consistent with the shorter half-life of 42 sec for E1-2,3-Q as compared to 12 min for E1-3,4-Q (Iverson et al., 1996). Overall, the model captures the joint action of the phase I and II enzymes rather well, allowing the simulation of the pathway from the parent hormone E₂ through several enzymatic steps to the most distal metabolites, the GSH-estrogen conjugates. Since these conjugates are produced via the quinones, the excellent agreement between simulated and measured GSH-estrogen conjugate levels provides further assurance about the validity of modeling the estrogen quinones.

Although other phase I enzymes, such as CYP1A2 and CYP3A4, are involved in hepatic and extrahepatic estrogen oxidation, CYP1A1 and 1B1 display the highest levels of expression in breast tissue and therefore are considered the principal oxidizing enzymes in mammary estrogen metabolism (Huang et al., 1996; Spink et al., 1998). COMT shows ubiquitous expression in all tissues including breast (Weisz et al., 2000). While COMT is the sole methylating enzyme, there are potentially three GSH-conjugating enzymes active in the pathway. Based on protein levels in breast tissue, GSTP1 is the predominant member of the GST family with GSTM1 and GSTA1 present at much lower levels (Cairns et al., 1992; Kelley et al., 1994; Alpert et al., 1997). GSTs are known to have selective as well as overlapping substrate specificities and it is presently not known whether GSTM1 and GSTA1 share with GSTP1 the ability to conjugate estrogen quinones (Hachey et al., 2003). In order to determine the potential roles of GSTM1 and GSTA1 in estrogen metabolism, the inventor plans to prepare each as purified, recombinant enzyme, followed by kinetic studies to define their respective rate constants. Besides COMT, there are two other classes of phase II enzymes capable of conjugating catechol estrogens, namely, the sulfotransferases (SULTs) and UDP-glucurunosyltransferases (UGTs). It appears that the catechol estrogens are converted predominantly to methyl conjugates and to a lesser extent sulfate and glucuronide conjugates (Raftogianis et al., 2000). In future experiments, the inventor will assess the role of SULTs and UGTs. The present mathematical model only incorporates the key phase I enzymes CYP1A1 and CYP1B1 and the phase II enzymes COMT and GSTP1. However, the model can readily accommodate additional enzymes and allow inclusion of other GST members as well as SULTs and UGTs in the same manner as currently is done for CYP1A1 and CYP1B1. In contrast to the complex kinetics of the methoxyestrogens, the sulfate and glucuronide conjugation reactions follow simple substrate-product kinetics like the GSTP1-mediated GSH conjugation. Therefore, straightforward modeling with good agreement of simulated and experimental data is anticipated.

Each of the phase I and II enzymes involved in estrogen metabolism possesses genetic variants that (1) are associated with altered enzyme function and (2) occur in a sizable portion of the population (Garte et al., 2001; Mitrunen and Hirvonen, 2003). Since the experimental analysis used only wild-type recombinant enzymes, the results provide a limited view of estrogen metabolism. To obtain a more realistic and inclusive view of estrogen metabolism in the female population, the inventor utilized the mathematical model to simulate how variations in the kinetic parameters resulting from polymorphisms of the enzymes impact the metabolite concentrations. Four 4 CYP1A1, 16 CYP1B1, and 2 COMT alleles were examined. GSTP1 also has two polymorphisms, i.e., 104Ile→Val and 113Ala→Val (Ali-Osman et al., 1997; Zimniak et al., 1994), but it is unknown whether they affect GSH-estrogen conjugation. Thus, the present simulations are based on the examination of 4×16×2 genetic combinations to demonstrate the utility of the model. Although each of the metabolites can be modeled, the inventor's analysis concentrated on the catechols and quinones because of their documented carcinogenic activity (Stack et al., 1996; Akanni and Abul-Hajj, 1997; Cavalieri et al., 2002; Bolton et al., 1998). As shown in FIGS. 3A-D, modeling of the 128 haplotype combinations produced a continuous spectrum of catechol and quinone concentrations over time, as expressed by a range of AUCs. The simulations identified the haplotype combinations producing the highest and lowest AUCs. For example, the maximum AUCs for 4-OHE₂ and E₂-3,4-Q were produced by the haplotype CYP1A1_{461Asn-462Ile}CYP1B1_{48Arg-119Ser-432Val-453Asn}COMT_{108Met}, which were 2.6- and 4.6-fold higher, respectively, than the minimum AUCs produced by haplotype CYP1A1_{461Thr-462Val}CYP1B1_{48Gly-119Ala-432Val-453Ser}COMT_{108Val}. While these differences may not appear large, it is important to consider that they impact on lifetime exposure, which is consistent with the hormonal risk model presented by Pike et al. (1983).

This kinetic-genomic model is pertinent to the numerous epidemiological studies that have examined the association of genetic variants of enzymes involved in estrogen metabolism with breast cancer risk (Mitrunen and Hirvonen, 2003; Dunning et al., 1999). These studies have been handicapped by investigating only one or two enzymes, but even those examining all enzymes have been fundamentally limited by not being able to assess the underlying metabolic interactions (Thomas, 2005). This model attempts to fill this gap and apply it to a hospital-based case-control population that has been analyzed previously with respect to CYP1A1, CYP1B1, and COMT genotypes (Ritchie et al., 2001; Bailey et al., 1998; Bailey et al., 1998). Here, the inventor went beyond genotypes and used the model to determine for each woman the effect of her composite CYP1A1, CYP1B1, and COMT haplotypes on estrogen metabolite production.

Inherited variations in enzyme genotype persist throughout life and can therefore be regarded as constants for each individual. However, the very same genes are also subject to induction and levels of enzyme expression may vary considerably as a result of the high degree of inducibility by a variety of agents. For example, cytochrome P450 enzymes are induced by hundreds of compounds (dietary and environmental chemicals, drugs) and human exposure to such xenobiotics is unavoidable (Conney, 2003). Intra- and interindividual variation in xenobiotic exposure has several consequences: (1) the P450 activity in an individual may change over time, (2) the P450 activity may differ between individuals of the same genotype, and (3) the phenotypic variability in P450 activity may be greater than the effect of genetic polymorphisms due to the strong inducing power of certain xenobiotics. Thus, while each individual has a unique composite E₂-3,4-Q AUC based on her subset of 128 genetic combinations, the AUC value can vary with the phenotype. In this model, the inventor attempted to incorporate both the certainty of the enzyme genotype and the ambiguity of the phenotype, the latter indicated by the changing ratio of phase I enzymes CYP1B1/CYP1A1 (FIGS. 4A-C). Although one must accept imprecise information about P450 activity in breast tissue, it can be assumed that the concentration of CYP1B1 is greater than that of CYP1A1 based on mRNA expression levels, higher levels of 4-OHE₂ than 2-OHE₂, and the observation that 2-OHE₂ is produced by both CYP isoforms, whereas 4-OHE₂ is formed only by CYP1B1 (Hayes et al., 1996; Hanna et al., 2000; Huang et al., 1996; Spink et al., 1998). Since the 4-OHE₂/2-OHE₂ ratio can be ˜3 and reach as high as ˜5 (Liehr and Ricci, 1996; Castagnetta et al., 2002; Rogan et al., 2003), the inventor varied the CYP1B1/CYP1A1 ratio in the model from 2 to 5. For CYP ratios >2, the model identified a top tier of E₂-3,4-Q AUCs with significantly increased numbers of breast cancer cases in the top percentiles (FIGS. 4B,C and Table 3), suggesting that E₂-3,4-Q AUC may be an indicator of breast cancer risk. The ranking order of E₂-3,4-Q AUCs is primarily determined by the enzyme genotype, i.e., the composite CYP1A1-CYP1B1-COMT haplotype of a subject. However, the AUC ranking is also affected by the enzyme phenotype and a change in CYP1B1/CYP1A1 ratio may lead to a different ranking of a subject in the population (FIGS. 4B,C). This is due to the fact that CYP1B1 and CYP1A1 catalyze different reactions in the metabolic pathway. Changing their ratio will have different results on the E₂-3,4-Q AUC for subjects with different composite haplotypes.

Estrogens have long been recognized as prime risk factor for the development of breast cancer, but their assessment has not progressed beyond traditional exposure data such as parity, age at menarche and menopause, etc. Here, the inventor presents a new approach that is based on the molecular analysis of mammary estrogen metabolism. The E₂-3,4-Q AUC is a plausible metabolic risk factor for breast cancer because E₂-3,4-Q has been identified as principal estrogen metabolite causing DNA adduct formation in experimental animals and E₂-3,4-Q-derived DNA adducts have been detected in human breast cancer tissues (Li et al., 2004; Liehr et al., 1986; Li and Li, 1987; Cavalieri et al., 1997; Embrechts et al., 2003; Rogan et al., 2003; Markushin et al., 2003). Whether the E₂-3,4-Q AUC is an independent risk factor, as suggested by the present analysis, will need to be confirmed by a larger separate study. The value of E₂-3,4-Q AUC as a new metabolic-genetic risk factor may yet be in its combination with traditional measures of endogenous and exogenous estrogen exposure. For example, one could estimate the overall exposure of a woman to E₂-3,4-Q AUC by taking into account (1) total years of menstruation or menopause age, (2) total pregnancy time, (3) years of menstruation before first full-term pregnancy, (4) body mass index, (5) dosage and duration of oral contraceptives, (6) dosage and duration of hormone replacement therapy. Altogether, one could derive an individualized risk factor of estrogen exposure for each woman that combines her reproductive life history with her unique genetic and metabolic traits. Data on traditional variables related to estrogen exposure were unfortunately not obtained for all subjects of the present study population, such as the control subject who had the highest E₂-3,4-Q AUC in the entire population (Table 3).

In summary, using experimentally determined rate constants, the inventor developed a mathematical model of mammary estrogen metabolism that allowed the kinetic simulation of E₂ and eight metabolites. The simulations showed excellent agreement with experimental results and provided a quantitative assessment of the metabolic interactions. The model permits the simulation of the carcinogenic estrogen quinones, whose transient nature prevents their direct quantitation. Using rate constants of genetic variants of CYP1A1, CYP1B1, and COMT, the model allows examination of the kinetic impact of enzyme polymorphisms on the entire pathway, including the identification of those haplotypes producing the largest amounts of catechols and quinones. The inventor conceptually addressed the ambiguity of phenotypic information about enzyme concentration by varying the CYP1B1/CYP1A1 ratio. Application of the model to a breast cancer case-control population defined E₂-3,4-Q AUC as a potential risk factor. The model identified a subset of women with an increased risk of breast cancer based on their enzyme haplotype and consequent E₂-3,4-Q production. The model offers for the first time the opportunity to combine genetic, metabolic, and lifetime exposure data in assessing estrogens as breast cancer risk factor.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. REFERENCES

The following references, to the extent that they provide exemplary procedural or other details supplementary to those set forth herein, are specifically incorporated herein by reference.
Abul-Hajj et al., J. Sterioid Biochem., 31:107-110, 1988.
Akanni et al., Chem. Res. Toxicol., 10:760-766, 1997.
Ali-Osman et al., J. Biol. Chem., 15:10004-10012, 1997.
Alpert et al., Clin. Cancer Res., 3:661-667, 1997.
Alsobrook et al., Am. J. Med. Genet., 114:116-120, 2002.
Bailey et al., Cancer Res., 58:5038-5041, 1998.
Bailey et al., Cancer Res., 58:65-70, 1998.
Bejjani et al., Am. J. Hum. Genet., 62:325-333, 1998.
Bejjani et al., Hum. Molec. Genet., 9:367-374, 2000.
Bolton et al., Chem. Res. Toxicol., 11:1113-1127, 1998.
Brahe et al., Am. J. Med. Genet., 24:203-204, 1986.
Brahe et al., Hum. Genet., 74:230-234, 1986.
Bray et al., Am. J. Hum. Genet., 73:152-161, 2003.
Brown et al., Proc. Nat. Acad. Sci., 73: 4628-4632, 1976.
Bucan et al., Hum. Molec. Genet., 2:1245-1252, 1993.
Cairns et al., J. Pathol., 166:19-25, 1992.
Cascorbi et al., Cancer Res., 56:4965-4969, 1996. Castagnetta et al., Clin. Cancer Res., 8:3146-3155, 2002.
Cavalieri et al., Carcinogenesis, 23:1071-7, 2002.
Cavalieri et al., Proc. Natl. Acad. Sci. USA, 94:10937-10942, 1997.
Chen et al., Clin. Res., 31: 456A, 1983.
Conney, Annu. Rev. Pharmacol. Toxicol., 43:1-30, 2003.
Corchero et al., Pharmacogenetics, 11: 1-6, 2001.
Cosma et al., J. Toxicol. Environ. Health, 40:309-316, 1993.
Costantino et al., J. Natl. Cancer Inst., 91:1541-1548, 1999.
Dawling et al., Cancer Res., 61:6716-6722, 2001.
Dawling et al., Cancer Res., 63:3127-3132, 2003.
Dawling et al., Chem. Res. Toxicol., 17:1258-1264, 2004.
Dunham et al., Lancet, 340:1361-1362, 1992.
Dunning et al., Cancer Epidemiol. Biomarkers Prev., 8:843-854, 1999.
Dwivedy et al., Chem. Res. Toxicol., 5:828-833, 1992.
Elston et al., Biometrics, 33:536-542, 1977.
Embrechts et al., J. Am. Soc. Mass. Spectrom., 14:482-491, 2003.
European Patent Appln. 320,308
European Patent Appln. 329 822
Excoffier and Slatkin, Mol. Biol. Evol., 12:921-927, 1995.
Floderus and Wetterberg, Clin. Genet., 19:392-395, 1981.
Floyd et al., Carcinogenesis, 11: 1447-1450, 1990.
Fodor et al., Science, 251:767-773, 1991.
Frisch et al., Molec. Psychiat., 6:243-245, 2001.
Frohman, In: PCR Protocols: A Guide To Methods And Applications, Academic Press, N.Y., 1990.
Gabrovsek et al., Am. J. Med. Genet., 124B:68-72, 2004.
Garte et al., Cancer Epidemiol. Biomarkers Prev., 10:1239-1248, 2001.
GB 2,202,328
Geisler et al., Am. J. Epidemiol., 154:95-105, 2001.
Gershon et al., Am. J. Hum. Genet., 33:136 A, 1981.
Gorman et al., Science, 221:551-553, 1983.
Gorman, Life Sciences, 36(22):8, 1993.
Grossman et al., Cytogenet. Cell Genet., 58:2048, 1991.
Grossman et al., Genomics, 12:822-825, 1992.
Gustavson et al., Clin. Genet., 22:22-24, 1982.
Gustavson et al., Clin. Genet., 4:279-280, 1973.
Hachey et al., Cancer Res., 63:8492-8499, 2003.
Hacia et al., Nature Genet., 14:441-449, 1996.
Han et al., Carinogenesis, 15:997-1000, 1994.
Han et al., Carinogenesis, 16:2571-2574, 1995.
Hanna et al., Cancer Res., 60:3440-3444, 2000.
Hayashi et al., J. Biochem., 110:407-411, 1991.
Hayes et al., Crit. Rev. Biochem. Mol. Biol., 30:445-600, 1995.
Hayes et al., Proc. Natl. Acad. Sci. USA, 93:9776-9781, 1996.
Hildebrand et al., Biochem. Biophys. Res. Commun., 130: 396-406, 1985a.
Hildebrand et al., Nucleic Acids Res., 13: 2009-2016, 1985b.
Holmstrom et al., Anal. Biochem. 209:278-283, 1993.
Huang et al., Carcinogenesis, 18:83-88, 1997.
Huang et al., Drug Metab. Disp., 24:899-905, 1996.
Innis et al., Proc. Natl. Acad. Sci. USA, 85(24):9436-9440, 1988.
Iverson et al., Chem. Res. Toxicol., 9:492-499, 1996.
Jaiswal et al, Nucleic Acids Res., 14: 4376, 1986.
Jaiswal et al., Molec. Endocr., 1: 312-320, 1987.
Jaiswal et al., Nucleic Acids Res., 13: 4503-4520, 1985.
Jaiswal et al., Nucleic Acids Res., 14: 6773-6774, 1986.
Jaiswal et al., Science, 228: 80-83, 1985.
Jaiswal, et al., J. Exp. Path., 3: 1-17, 1987.
Jiang et al., Hum. Mutat., 25: 196-206, 2005.
Jones et al., Nucleic Acids Res., 19: 6547-6551, 1991.
Kalyanaraman et al., J. Biol. Chem., 259:14018-14022, 1984.
Karayiorgou et al., Biol. Psychiat., 45:1178-1189, 1999.
Karayiorgou et al., Proc. Nat. Acad. Sci., 94:4572-4575, 1997.
Kawajiri et al., FEBS Lett., 263: 131-133, 1990.
Kawajiri et al., Cancer Res., 56: 72-76, 1996.
Kawajiri et al., Europ. J. Biochem., 159: 219-225, 1986.
Kelley et al., Biochem. J, 304:843-848, 1994.
Kouri et al., Cancer Res., 42: 5030-5037, 1982.
Kwoh et al., Proc. Natl. Acad. Sci. USA, 86:1173, 1989.
Lachman et al., 1996
Lee et al., Hum. Genet., 116:319-328, 2005.
Levitt and Baron, Sixth Int. Cong. Hum. Genet., Jerusalem, 21, 1981.
Lewis et al., Chem-Biol. Interact., 145:281-295, 2003.
Li and Li, Fed. Proc., 46:1858-1663, 1987.
Li et al., Carcinogenesis, 25:289-297, 2004.
Liehr et al., Radical Biol. Med., 8:415-423, 1990.
Liehr et al., J. Steroid Biochem., 24:353-356, 1986.
Liehr and Ricci, Proc. Natl. Acad. Sci. USA, 93:3294-3296, 1996.
Liu and Muse, www.powermarker.net.
Lundstrom et al., DNA Cell Biol., 10:181-189, 1991.
Mannervik et al., Biochem. J, 282:305-308, 1992.
Markushin et al., Chem. Res. Toxicol., 16:1107-1117, 2003.
McTieman et al., Cancer Epidemiol. Biomarkers Prev., 10:333-338, 2001.
Melki et al., J. Med. Genet., 41:647-651, 2004.
Michaelovsky et al., Am. J. Med. Genet., 139B:45-50, 2005.
Ming and Muenke, Am. J. Hum. Genet., 71:1017-1032, 2002.
Mitrunen and Hirvonen, Mutation Res., 544:9-41, 2003.
Mooney et al., Carcinogenesis, 18: 503-509, 1997.
Nakachi et al., Cancer Res., 51: 5177-5180, 1991.
Nandi et al., Proc. Natl. Acad. Sci. USA, 92:3650-3657, 1995.
Nebert and Gonzalez, Annu. Rev. Biochem., 56: 945-993, 1987.
Newbold and Liehr, Cancer Res., 60:235-237, 2000.
Newton et al., Nucl. Acids Res. 21:1155-1162, 1993.
Ocraft et al., Ann. Hum. Genet., 49: 237-239, 1985.
Ohara et al., Proc. Natl. Acad. Sci. USA, 86:5673-5677, 1989.
Palmatier et al., Molec. Psychiat., 9:859-870, 2004.
Paolini et al., Nature, 398: 760-761, 1999.
PCT Appln. PCT/US87/00880
PCT Appln. PCT/US89/01025
PCT Appln. WO 88/10315
PCT Appln. WO 89/06700
PCT Appln. WO 90/07641
Pease et al., Proc. Natl. Acad. Sci. USA, 91:5022-5026, 1994.
Perera, Science, 278: 1068-1073, 1997.
Persson et al., Biochem. Biophys. Res. Commn., 231:227-230, 1997.
Petersen et al., Am. J. Hum. Genet., 48: 720-725, 1991.
Pike et al., Nature, 303:767-770, 1983.
Quattrochi et al., DNA, 4: 395-400, 1985.
Raftogianis et al., J. Natl. Cancer Inst. Monogr., 27:113-124, 2000.
Rasmussen et al., Anal. Biochem, 198:138-142, 1991.
Reddy and Chow, Am. J. Health Syst Pharm., 57:1315-2132, 2000.
Ritchie et al., Am. J. Hum. Genet., 69:138-147, 2001.
Rogan et al., Carcinogenesis, 24:697-702, 2003.
Roy et al., Carcinogenesis, 11:459-462, 1990.
Running et al., BioTechniques 8:276-277, 1990.
Sambrook and Russell, Molecular Cloning: A Laboratory Manual 3^{rd} Ed., Cold Spring Harbor Laboratory Press, 2001.
Scanlon et al., Science, 203:63-65, 1979.
Schwartzman et al., Proc. Nat. Acad. Sci, USA, 84: 8125-8129, 1987.
Seidegard et al., Proc. Nat. Acad. Sci. USA, 85:7293-7297, 1988.
Shifman et al., Am. J. Hum. Genet., 71:296-1302, 2002.
Shimada et al., Cancer Res., 56:2979-2984, 1996.
Shoemaker et al., Nature Genetics, 14:450-456, 1996.
Shprintzen et al., Am. J. Med. Genet., 42:141-142, 1992.
Spielman and Weinshilboum, Am. J. Med. Genet., 10:279-290, 1981.
Spink et al., Arch. Biochem. Biophys., 293:342-348, 1992.
Spink et al., Biochem., Mol. Biol., 51:251-258, 1994.
Spink et al., Carcinogenesis, 19:291-298, 1998.
Stack et al., Chem. Res. Toxicol., 9:851-859, 1996.
Stoilov et al., Am. J. Hum. Genet., 62:573-584, 1998.
Stoilov et al., Hum. Molec. Genet., 6: 641-647, 1997.
Strange et al., Mutat. Res., 482:21-26, 2001.
Sutter et al., J. Biol. Chem., 269:13092-13099, 1994.
Sweet et al., Molec. Psychiat., 10:1026-1036, 2005.
Syvanen et al., Pharmacogenetics, 765-71, 1997.
Tabakovic and Abul-Hajj, Chem. Res. Toxicol., 7:696-701, 1994.
Tabakovic et al., Chem. Res. Toxicol., 9:860-865, 1996.
Tang et al., J. Biol. Chem., 271:28324-28330, 1996.
Thomas, Cancer Epidemiol. Biomarkers Prev., 14:557-559, 2005.
Thum and Borlak, Lancet, 355: 979-983, 2000.
Tukey et al., Proc. Natl. Acad. Sci. USA, 81:3163-3166, 1984.
U.S. Pat. No. 4,683,195
U.S. Pat. No. 4,683,202
U.S. Pat. No. 4,800,159
U.S. Pat. No. 4,883,750
U.S. Pat. No. 5,578,832
U.S. Pat. No. 5,837,832
U.S. Pat. No. 5,837,860
U.S. Pat. No. 5,861,242
U.S. Pat. No. 6,159,693
U.S. Publn. 2005/0255504
Vincent et al., Am. J. Hum. Genet., 70:448-460, 2002.
Vincent et al., J. Med. Genet., 38:324-326, 2001.
Walker et al., Nucleic Acids Res. 20(7):1691-1696, 1992.
Wang et al., JAMA, 287(2):195-202, 2002.
Weinshilboum and Dunnette, Clin. Genet., 19:426-437, 1981.
Weinshilboum and Raymond, Am. J. Hum. Genet., 29:125-135, 1977.
Weisz et al., Am. J. Pathol., 156:1841-18448, 2000.
Wiencke et al., Cancer Res., 55(21):4910-4914. 1995.
Wilson et al., Am. J. Med. Genet., 19:525-532, 1984.
Winqvist et al., Cytogenet. Cell Genet., 58:2051, 1991.
Wu et al., Proc. Natl. Acad. Sci. USA, 86(8):2757-2760, 1989.
Xu et al., J. Biol. Chem., 273:3517-3527, 1998.
Yager and Liehr, Annu. Rev. Pharmacol. Toxicol., 36:203-232, 1996.
Ye et al., Hum. Mutat., 17(4):305-16, 2001.
Zaykin et al., Hum. Hered., 53:79-91, 2002.
Zhang et al., Cancer Res., 56:3926-3933, 1996.
Zimniak et al., Eur. J. Biochem., 224:893-899, 1994.

What is claimed is:

1. A method for assessing a female subject's risk for developing breast cancer comprising:
(a) determining, in a sample from said subject, the allelic profile of COMT, CYP1A1 and CYP1B1; and
(b) predicting, based an in silico model of estrogen biosynthesis, relative amounts of 4-OHE₂ and/or E2-3,4-Q produced by the determined allelic profile,
wherein increased risk of developing breast cancer is associated with increased production of 4-OHE₂ and/or E₂-3,4-Q as compared to mean production by a relevant genetic population, and reduced risk of developing breast cancer is associated with reduced production of 4-OHE₂ and/or E₂-3,4-Q as compared to mean production by a relevant genetic population.

2. The method of claim 1, wherein increased risk is associated with increased production of E₂-3,4-Q.

3. The method of claim 1, wherein increased risk is associated with increased production of 4-OHE₂ and E₂-3,4-Q.

4. The method of claim 1, wherein decreased risk is associated with decreased production of E₂-3,4-Q.

5. The method of claim 1, wherein decreased risk is associated with decreased production of 4-OHE₂ and E₂-3,4-Q.

6. The method of claim 1, wherein said model adjusts the relative ratio of CYP1B1/CYP1A1.

7. The method of claim 6, wherein said ratio is adjusted to 5:1.

8. The method of claim 1, further comprising assessing one or more aspects of the subject's personal history.

9. The method of claim 1, wherein said one or more aspects are selected from the group consisting of age, ethnicity, reproductive history, menstruation history, use of oral contraceptives, body mass index, alcohol consumption history, smoking history, exercise history, diet, family history of breast cancer or other cancer including the age of the relative at the time of their cancer diagnosis, and a personal history of breast cancer, breast biopsy or DCIS, LCIS, or atypical hyperplasia.

10. The method of claim 8, wherein one or more aspects comprises age.

11. The method of claim 1, wherein determining said allelic profile is achieved by amplification of nucleic acid from said sample.

12. The method of claim 11, wherein amplification comprises PCR.

13. The method of claim 11, wherein primers for amplification are located on a chip.

14. The method of claim 11, wherein primers for amplification are specific for alleles of said genes.

15. The method of claim 11, further comprising cleaving amplified nucleic acid.

16. The method of claim 1, wherein said sample is derived from oral tissue or blood.

17. The method of claim 1, further comprising making a decision on the timing and/or frequency of cancer diagnostic testing for said subject.

18. The method of claim 1, further comprising making a decision on the timing and/or frequency of prophylactic cancer treatment for said subject.

19. A method for determining the need for routine diagnostic testing of a female subject for breast cancer comprising:
(a) determining, in a sample from said subject, the allelic profile of COMT, CYP1A1 and CYP1B1; and
(b) predicting, based an in silico model of estrogen biosynthesis, relative amounts of 4-OHE₂ and/or E₂-3,4-Q produced by the determined allelic profile,
wherein need for routine diagnostic testing is associated with increased production of 4-OHE₂ and/or E₂-3,4-Q as compared to mean production by a relevant genetic population.

20. The method of claim 19, wherein need for routine testing is associated with increased production of E₂-3,4-Q.

21. The method of claim 19, wherein need for routine testing is associated with increased production of 4-OHE₂ and E₂-3,4-Q.

22. A method for determining the need of a female subject for prophylactic anti-breast cancer therapy comprising:
(a) determining, in a sample from said subject, the allelic profile of COMT, CYP1A1 and CYP1B1; and
(b) predicting, based an in silico model of estrogen biosynthesis, relative amounts of 4-OHE₂ and/or E₂-3,4-Q produced by the determined allelic profile,
wherein need for prophylactic breast cancer therapy is associated with increased production of 4-OHE₂ and/or E₂-3,4-Q as compared to mean production by a relevant genetic population.

23. The method of claim 22, wherein need for prophylactic breast cancer therapy is associated with increased production of E₂-3,4-Q.

24. The method of claim 22, wherein need for prophylactic breast cancer therapy is associated with increased production of 4-OHE₂ and E₂-3,4-Q.


SEQUENCE LISTING (6 sequences)

SEQ ID NO: 1 - DNA, 2602 bp, Homo sapiens
  CDS (123)..(1661)
ctcaccctgaaggtgacagttccttggaaccttccctgatccttgtgatcccaggctccaagagtccacccttcccagctcagctcagtacctcagccacctccaagatccctacactgatcatgcttttcccaatctccatgtcggccacggagtttcttctggcctctgtcatcttctgtctggtattctgggtaatcagggcctcaagacctcaggtccccaaaggcctgaagaatccaccagggccatggggctggcctctgattgggcacatgctgaccctgggaaagaacccgcacctggcactgtcaaggatgagccagcagtatggggacgtgctgcagatccgaattggctccacacccgtggtggtgctgagcggcctggacaccatccggcaggccctggtgcggcagggcgatgatttcaagggccggcccgacctctacaccttcaccctcatcagtaatggtcagagcatgtccttcagcccagactctggaccagtgtgggctgcccgccggcgcctggcccagaatggcctgaaaagtttctccattgcctctgacccagcctcctcaacctcctgctacctggaagagcatgtgagcaaggaggctgaggtcctgataagcacgttgcaggagctgatggcagggcctgggcactttaacccctacaggtatgtggtggtatcagtgaccaatgtcatctgtgccatttgctttggccggcgctatgaccacaaccaccaagaactgcttagcctagtcaacctgaataataatttcggggaggtggttggctctggaaacccagctgacttcatccctattcttcgctacctacccaacccttccctgaatgccttcaaggacctgaatgagaagttctacagcttcatgcagaagatggtcaaggagcactacaaaacctttgagaagggccacatccgggacatcacagacagcctgattgagcactgtcaggagaagcagctggatgagaacgccaatgtccagctgtcagatgagaagatcattaacatcgtcttggacctctttggagctgggtttgacacagtcacaactgctatctcctggagcctcatgtatttggtgatgaaccccagggtacagagaaagatccaagaggagctagacacagtgattggcaggtcacggcggccccggctctctgacagatcccatctgccctatatggaggccttcatcctggagaccttccgacactcttccttcgtccccttcaccatcccccacagcacaacaagagacacaagtttgaaaggcttttacatccccaaggggcgttgtgtctttgtaaaccagtggcagatcaaccatgaccagaagctatgggtcaacccatctgagttcctacctgaacggtttctcacccctgatggtgctatcgacaaggtgttaagtgagaaggtgattatctttggcatgggcaagcggaagtgtatcggtgagaccattgcccgctgggaggtctttctcttcctggctatcctgctgcaacgggtggaattcagcgtgccactgggcgtgaaggtggacatgacccccatctatgggctaaccatgaagcatgcctgctgtgagcacttccaaatgcagctgcgctcttaggtgcttgagagccctgaggcctagactctgtctacctggtctggttgggcagccagaccagcaggctggcctatgtggtctaagattcagcctgaaactcatagacactgatctggctgcagttttgctatctgggctgtgggcaagcctaagggatcctgcctgcccctaccctggacttgcctctgcacaccctccagagacaacaggtaaaacagggccacatagatgctgatggagccttcccaagttgtgcttgagccaggaggcctgctagggttaggaggtccttaggcctctgagaagctctgaagaactctctggaagcccctgggcccagtacctagctggctctgtgagggtgctgactggcttcagcaagttagaactagccaaaccaggaccctgtccaatctttgacaattgggagctgccaagagtgaagggaagagacagcccaggatactggcacagaggtagtctcactgcttgaactaggctgagcaatctgaccctatgggtctaggacacagttcctgggaacatcacattcctctgcccttcctgcaggcaggaacaaacagggctgccttctggccttgtaagacccttattgctgtcctggaggggctggggacttgtgtctgcggggatccagagcgcacagggagtgcacatatccaggcaccaggactagggctggagtgaggggggggtatttcaattaccttctattggtctcccttctctacactcttgtaataaaatgtctatttttaatgtttgtacacaacaatccttctattctagcctgcattgagcttgcatgcttgcataagagcttaagaaccattgatttaatgtaatagggaaaattctaacccaggtatccaaaaatgtgtaagaacaactacctgagctaaataaagatattgttcaga

SEQ ID NO: 2 - protein, 512 aa, Homo sapiens [sequence omitted]

SEQ ID NO: 3 - DNA, 5128 bp, Homo sapiens
  CDS (373)..(2004)
actctggagtgggagtgggagcgagcgcttctgcgactccagttgtgagagccgcaagggcatgggaattgacgccactcaccgacccccagtctcaatctcaacgctgtgaggaaacctcgactttgccaggtccccaagggcagcggggctcggcgagcgaggcacccttctccgtccccatcccaatccaagcgctcctggcactgacgacgccaagagactcgagtgggagttaaagcttccagtgagggcagcaggtgtccaggccgggcctgcgggttcctgttgacgtcttgccctaggcaaaggtcccagttccttctcggagccggctgtcccgcgccactggaaaccgcacctccccgcagcatgggcaccagcctcagcccgaacgacccttggccgctaaacccgctgtccatccagcagaccacgctcctgctactcctgtcggtgctggccactgtgcatgtgggccagcggctgctgaggcaacggaggcggcagctccggtccgcgcccccgggcccgtttgcgtggccactgatcggaaacgcggcggcggtgggccaggcggctcacctctcgttcgctcgcctggcgcggcgctacggcgacgttttccagatccgcctgggcagctgccccatagtggtgctgaatggcgagcgcgccatccaccaggccctggtgcagcagggctcggccttcgccgaccggccggccttcgcctccttccgtgtggtgtccggcggccgcagcatggctttcggccactactcggagcactggaaggtgcagcggcgcgcagcccacagcatgatgcgcaacttcttcacgcgccagccgcgcagccgccaagtcctcgagggccacgtgctgagcgaggcgcgcgagctggtggcgctgctggtgcgcggcagcgcggacggcgccttcctcgacccgaggccgctgaccgtcgtggccgtggccaacgtcatgagtgccgtgtgtttcggctgccgctacagccacgacgaccccgagttccgtgagctgctcagccacaacgaagagttcgggcgcacggtgggcgcgggcagcctggtggacgtgatgccctggctgcagtacttccccaacccggtgcgcaccgttttccgcgaattcgagcagctcaaccgcaacttcagcaacttcatcctggacaagttcttgaggcactgcgaaagccttcggcccggggccgccccccgcgacatgatggacgcctttatcctctctgcggaaaagaaggcggccggggactcgcacggtggtggcgcgcggctggatttggagaacgtaccggccactatcactgacatcttcggcgccagccaggacaccctgtccaccgcgctgcagtggctgctcctcctcttcaccaggtatcctgatgtgcagactcgagtgcaggcagaattggatcaggtcgtggggagggaccgtctgccttgtatgggtgaccagcccaacctgccctatgtcctggccttcctttatgaagccatgcgcttctccagctttgtgcctgtcactattcctcatgccaccactgccaacacctctgtcttgggctaccacattcccaaggacactgtggtttttgtcaaccagtggtctgtgaatcatgacccagtgaagtggcctaacccggagaactttgatccagctcgattcttggacaaggatggcctcatcaacaaggacctgaccagcagagtgatgattttttcagtgggcaaaaggcggtgcattggcgaagaactttctaagatgcagctttttctcttcatctccatcctggctcaccagtgcgatttcagggccaacccaaatgagcctgcgaaaatgaatttcagttatggtctaaccattaaacccaagtcatttaaagtcaatgtcactctcagagagtccatggagctccttgatagtgctgtccaaaatttacaagccaaggaaacttgccaataagaagcaagaggcaagctgaaattttagaaatattcacatcttcggagatgaggagtaaaattcagtttttttccagttcctcttttgtgctgcttctcaattagcgtttaaggtgagcataaatcaactgtccatcaggtgaggtgtgctccatacccagcggttcttcatgagtagtgggctatgcaggagcttctgggagatttttttgagtcaaagacttaaagggcccaatgaattattatatacatactgcatcttggttatttctgaaggtagcattctttggagttaaaatgcacatatagacacatacacccaaacacttacaccaaactactgaatgaagaagtattttggtaaccaggccatttttggtgggaatccaagattggtctcccatatgcagaaatagacaaaaagtatattaaacaaagtttcagagtatattgttgaagagacagagacaagtaatttcagtgtaaagtgtgtgattgaaggtgataagggaaaagataaagaccagaaattcccttttcaccttttcaggaaaataacttagactctagtatttatgggtggatttatccttttgccttctggtatacttccttacttttaaggataaatcataaagtcagttgctcaaaaagaaatcaatagttgaattagtgagtatagtggggttccatgagttatcatgaattttaaagtatgcattattaaattgtaaaactccaaggtgatgttgtacctcttttgcttgccaaagtacagaatttgaattatcagcaaagaaaaaaaaaaaagccagccaagctttaaattatgtgaccataatgtactgatttcagtaagtctcataggttaaaaaaaaaagtcaccaaatagtgtgaaatatattacttaactgtccgtaagcagtatattagtattatcttgttcaggaaaaggttgaataatatatgccttgtgtaatattgaaaattgaaaagtacaactaacgcaaccaagtgtgctaaaaatgagcttgattaaatcaaccacctatttttgacatggaaatgaagcagggtttcttttcttcactcaaattttggcgaatctcaaaattagatcctaagatgtgttcttatttttataacatctttattgaaattctatttataatacagaatcttgttttgaaaataacctaattaatatattaaaattccaaattcatggcatgcttaaattttaactaaattttaaagccattctgattattgagttccagttgaagttagtggaaatctgaacattctcctgtggaaggcagagaaatctaagctgtgtctgcccaatgaataatggaaaatgccatgaattacctggatgttctttttacgaggtgacaagagttggggacagaactcccattacaactgaccaagtttctcttctagatgattttttgaaagttaacattaatgcctgctttttggaaagtcagaatcagaagatagtcttggaagctgtttggaaaagacagtggagatgaggtcagttgtgttttttaagatggcaattactttggtagctgggaaagcataaagctcaaatgaaatgtatgcattcacatttagaaaagtgaattgaagtttcaagttttaaagttcattgcaattaaacttccaaagaaagttctacagtgtcctaagtgctaagtgcttattacattttattaagctttttggaatctttgtaccaaaattttaaaaaagggagtttttgatagttgtgtgtatgtgtgtgtggggtggggggatggtaagagaaaagagagaaacactgaaaagaaggaaagatggttaaacattttcccactcattctgaattaattaatttggagcacaaaattcaaagcatggacatttagaagaaagatgtttggcgtagcagagttaaatctcaaataggctattaaaaaagtctacaacatagcagatctgttttgtggtttggaatattaaaaaacttcatgtaattttattttaaaatttcatagctgtacttcttgaatataaaaaatcatgccagtatttttaaaggcattagagtcaactacacaaagcaggcttgcccagtacatttaaattttttggcacttgccattccaaaatattatgccccaccaaggctgagacagtgaatttgggctgctgtagcctatttttttagattgagaaatgtgtagctgcaaaaataatcatgaaccaatctggatgcctcattatgtcaaccaggtccagatgtgctataatctgtttttacgtatgtaggcccagtcgtcatcagatgcttgcggcaaaagaaagctgtgtttatatggaagaaagtaaggtgcttggagtttacctggcttatttaatatgcttataacctagttaaagaaaggaaaagaaaacaaaaaacgaatgaaaataactgaatttggaggctggagtaatcagattactgctttaatcagaaaccctcattgtgtttctaccggagagagaatgtatttgctgacaaccattaaagtcagaagttttactccaggttattgcaataaagtataatgtttattaaatgcttcatttgtatgtcaaagctttgactctataagcaaattgcttttttccaaaacaaaaagatgtctcaggtttgttttgtgaattttctaaaagctttcatgtcccagaacttagcctttacctgtgaagtgttactacagccttaatattttcctagtagatctatattagatcaaatagttgcatagcagtatatgttaatttgtgtgtttttagctgtgacacaactgtgtgattaaaaggtatactttagtagacatttataactcaaggataccttcttatttaatcttttcttatttttgtactttatcatgaatgcttttagtgtgtgcataatagctacagtgcatagttgtagacaaagtacattctggggaaacaacatttatatgtagcctttactgtttgatataccaaattaaaaaaaaattgtatctcattacttatactgggacaccattaccaaaataataaaaatcactttcataatcttgaaaaaa

SEQ ID NO: 4 - protein, 543 aa, Homo sapiens
MGTSLSPNDPWPLNPLSIQQTTLLLLLSVLATVHVGQRLLRQRRRQLRSAPPGPFAWPLIGNAAAVGQAAHLSFARLARRYGDVFQIRLGSCPIVVLNGERAIHQALVQQGSAFADRPAFASFRVVSGGRSMAFGHYSEHWKVQRRAAHSMMRNFFTRQPRSRQVLEGHVLSEARELVALLVRGSADGAFLDPRPLTVVAVANVMSAVCFGCRYSHDDPEFRELLSHNEEFGRTVGAGSLVDVMPWLQYFPNPVRTVFREFEQLNRNFSNFILDKFLRHCESLRPGAAPRDMMDAFILSAEKKAAGDSHGGGARLDLENVPATITDIFGASQDTLSTALQWLLLLFTRYPDVQTRVQAELDQVVGRDRLPCMGDQPNLPYVLAFLYEAMRFSSFVPVTIPHATTANTSVLGYHIPKDTVVFVNQWSVNHDPVKWPNPENFDPARFLDKDGLINKDLTSRVMIFSVGKRRCIGEELSKMQLFLFISILAHQCDFRANPNEPAKMNFSYGLTIKPKSFKVNVTLRESMELLDSAVQNLQAKETCQ

SEQ ID NO: 5 - DNA, 816 bp, Artificial Sequence
  Description of Artificial Sequence  Synthetic Construct
atgccggaggccccgcctctgctgttggcagctgtgttgctgggcctggtgctgctggtggtgctgctgctgcttctgaggcactggggctggggcctgtgccttatcggctggaacgagttcatcctgcagcccatccacaacctgctcatgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggcaagatcgtggacgccgtgattcaggagcaccagccctccgtgctgctggagctgggggcctactgtggctactcagctgtgcgcatggcccgcctgctgtcaccaggggcgaggctgatcaccatcgagatcaaccccgactgtgccgccatcacccagcggatggtggatttcgctggcgtgaaggacaaggtcacccttgtggttggagcgtcccaggacatcatcccccagctgaagaagaagtatgatgtggacacactggacatggtcttcctcgaccactggaaggaccggtacctgccggacacgcttctcttggaggaatgtggcctgctgcggaaggggacagtgctactggctgacaacgtgatctgcccaggtgcgccagacttcctagcacacgtgcgcgggagcagctgctttgagtgcacacactaccaatcgttcctggaatacagggaggtggtggacggcctggagaaggccatctacaagggcccaggcagcgaagcagggccctag

SEQ ID NO: 6 - protein, 271 aa, Homo sapiens
MPEAPPLLLAAVLLGLVLLVVLLLLLRHWGWGLCLIGWNEFILQPIHNLLMGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGVKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP